(19) United States
(12) Patent Application Publication
(10) Pub. No.: US 2025/0308632 A1
(43) Pub. Date: Oct. 2, 2025

(54) PROGNOSIS AND TREATMENT OF MOLECULAR SUBTYPES OF PROSTATE CANCER

(71) Applicant: Veracyte, Inc., South San Francisco, CA (US)

(72) Inventors: Elai Davicioni, San Diego, CA (US); Yang Liu, Vancouver (CA)

(21) Appl. No.: 18/864,879

(22) PCT Filed: May 11, 2023
(86) PCT No.: PCT/US2023/021944

§ 371 Date: Nov. 11, 2024

Related U.S. Application Data
(60) Provisional application No. 63/341,367, filed on May 12, 2022.

(51) Int. Cl. G16B 25/10; G16B 40/20; G16H 10/60; G16H 20/10; G16H 20/40; G16H 50/20

(52) U.S. Cl. CPC G16B 25/10; G16B 40/20; G16H 10/60; G16H 20/10; G16H 20/40; G16H 50/20

(57) ABSTRACT

The present disclosure relates to methods, systems and kits for the diagnosis, prognosis and the determination of cancer progression of cancer in a subject. The disclosure also provides biomarkers that define subgroups of prostate cancer, clinically useful classifiers for distinguishing prostate cancer subtypes, bioinformatic methods for determining clinically useful subtyping classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

### CROSS-REFERENCE TO RELATED APPLICATIONS

This application claims priority to U.S. Ser. No. 63/341,367 filed May 12, 2022, which is herein incorporated by reference in its entirety.

### FIELD

The present disclosure relates to methods, systems and kits for the diagnosis, prognosis and the treatment of molecular subtypes of prostate cancer in a subject. The disclosure also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer and response to therapy based on molecular subtyping are also provided.

### BACKGROUND

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, prostate cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if prostate cancer cells spread to a bone (or anywhere else), it can mean that the individual has metastatic prostate cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

The present disclosure relates to methods, systems and kits for the diagnosis, prognosis and the determination of cancer progression of cancer in a subject. The disclosure also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

In some embodiments, the present disclosure provides a method comprising: optionally providing, obtaining, or having obtained a biological sample from a prostate cancer subject; performing or having performed an assay to detect or determine the presence and/or expression level of at least one or more targets selected from Table 2 in a sample from a prostate cancer subject; and administering a treatment to the subject, wherein the treatment is selected from the group consisting of surgery, chemotherapy, radiation therapy, immunotherapy/biological therapy, hormonal therapy, and photodynamic therapy based at least in part on the presence and/or expression level of the at least one or more genes selected from Table 2. In some embodiments, the present disclosure provides a method of subtyping prostate cancer in a subject, comprising: optionally providing, obtaining, or having obtained, a biological sample comprising prostate cancer cells from the subject, and performing or having performed an assay to detect or determine the presence and/or level of expression of at least one or more targets selected from Table 2 using at least one reagent that specifically binds to said targets; wherein the alteration of said expression level provides an indication of the prostate cancer subtype. In some embodiments, the alteration in the expression level of said target is reduced expression of said target. In other embodiments, the alteration in the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In still other embodiments, the target comprises a nucleic acid sequence. In some embodiments, the prostate cancer subtype is selected from the group consisting of Luminal Differentiated (LD), Luminal Proliferating (LP), Basal Immune (BI), and Basal Neuroendocrine-like (BN). In other embodiments, the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, and/or celecoxicib.

In some embodiments, the present disclosure also provides a method of diagnosing, prognosing, assessing the risk of recurrence or predicting benefit from therapy in a subject with prostate cancer, comprising: optionally providing, obtaining, or having obtained a biological sample comprising prostate cancer cells from the subject; performing or having performed an assay to determine or detect the presence and/or expression level in the biological sample from the subject for a plurality of targets using at least one reagent that specifically binds to said targets, wherein the plurality of targets comprises one or more targets selected from Table 2; and diagnosing, prognosing, assessing the risk of recurrence or predicting benefit from therapy in the subject based on the presence and/or expression levels of the plurality of targets. In some embodiments, the expression level of the target is reduced expression of the target. In other embodiments, the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In other embodiments, the target comprises a nucleic acid sequence. In some embodiments, the nucleic acid probe is a reporter probe and/or a capture probe.

In some embodiments, the present disclosure provides a system for analyzing a cancer, comprising, a probe set comprising a plurality of target sequences, wherein the plurality of target sequences hybridizes to one or more targets selected from Table 2; or the plurality of target sequences comprises one or more targets selected from Table 2; and a computer model or algorithm for analyzing an expression level and/or expression profile of the target hybridized to the probe in a sample from a subject suffering from prostate cancer. In some embodiments, the method further comprises a label that specifically binds to the target, the probe, or a combination thereof.

In some embodiments, the present disclosure provides a method comprising: (a) optionally providing, obtaining, or having obtained a biological sample from a subject with prostate cancer; (b) performing or having performed an assay to determine or detect the presence and/or expression level in the biological sample for a plurality of targets, wherein the plurality of targets comprises one or more targets selected from Table 2; (c) subtyping or obtaining the subtype of the prostate cancer in the subject based on the presence and/or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy based at least in part on the subtype of the prostate cancer. In some embodiments, the present disclosure provides a method of treating a subject with prostate cancer, comprising: optionally providing, obtaining, or having obtained a biological sample comprising prostate cancer cells from the subject; performing or having performed an assay to detect or determine the presence and/or level of expression of at least one or more targets selected from Table 2 using at least one reagent that specifically binds to said targets; subtyping or obtaining the subtype of the prostate cancer based on the level of expression or amplification of the at least one or more targets; and prescribing a treatment regimen based at least in part on the prostate cancer subtype. In some embodiments, the prostate cancer subtype is selected from the group consisting of Luminal Differentiated (LD), Luminal Proliferating (LP), Basal Immune (BI), and Basal Neuroendocrine-like (BN). In other embodiments, the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, and/or celecoxicib.

In some embodiments, the present disclosure provides a kit for analyzing a prostate cancer, comprising, a probe set comprising a plurality of target sequences, wherein the plurality of target sequences comprises at least one target sequence listed in Table 2; and a computer model or algorithm for analyzing an expression level and/or expression profile of the target sequences in a sample. In some embodiments, the method further comprises a computer model or algorithm for correlating the expression level or expression profile with disease state or outcome. In other embodiments, the method further comprises a computer model or algorithm for designating a treatment modality for the individual. In yet other embodiments, the method further comprises a computer model or algorithm for normalizing expression level or expression profile of the target sequences. In some embodiments, the method further comprises sequencing the plurality of targets. In some embodiments, the method further comprises hybridizing the plurality of targets to a solid support. In some embodiments, the solid support is a bead or array. In some embodiments, performing or having performed an assay of the expression level of a plurality of targets may comprise the use of a probe set. In some embodiments, performing or having performed an assay of the expression level may comprise the use of a classifier. The classifier may comprise a probe selection region (PSR). In some embodiments, the classifier may comprise the use of an algorithm. The algorithm may comprise a machine learning algorithm. In some embodiments, performing or having performed an assay of the expression level may also comprise sequencing the plurality of targets.

Disclosed herein methods for molecular subtyping of prostate cancer, wherein the subtypes have an AUC value of at least about 0.40 to predict patient outcomes. In some embodiments, patient outcomes are selected from the group consisting of biochemical recurrence (BCR), metastasis (MET) and prostate cancer death (PCSM) after radical prostatectomy. The AUC of the subtype may be at least about 0.40, 0.45, 0.50, 0.55, 0.60, 0.61, 0.62, 0.63, 0.64, 0.65, 0.66, 0.67, 0.68, 0.69, 0.70 or more.

Disclosed herein is a method for subtyping a prostate cancer, comprising determining the level of expression or amplification of at least one or more targets of the present disclosure, wherein the significance of the expression level of the one or more targets is based on one or more metrics selected from the group comprising T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The molecular subtypes of the present disclosure are useful for predicting clinical characteristics of subjects with prostate cancer. In some embodiments, the clinical characteristics are selected from the group consisting of seminal vesical invasion (SVI), lymph node invasion (LNI), prostate-specific antigen (PSA), and Gleason score (GS).

In some embodiments, the disclosure provides methods comprising: a) optionally providing, obtaining, or having obtained a biological sample from a subject having prostate cancer; b) measuring or obtaining a measure of the levels of expression in the biological sample of a plurality of genes selected from Table 2; and c) subtyping the prostate cancer of the subject according to a genomic subtyping classifier based on the levels of expression of the plurality of genes, wherein said subtyping comprises assigning the prostate cancer to one of four subtypes selected from the group consisting of Luminal Differentiated (LD), Luminal Proliferating (LP), Basal Immune (BI), and Basal Neuroendocrine-like (BN). In some embodiments, the methods further comprise administering a treatment to the subject, wherein the treatment is selected from the group consisting of surgery, chemotherapy, radiation therapy, immunotherapy/biological therapy, hormonal therapy, and photodynamic therapy based at least in part on the subtype of the cancer. In yet other embodiments, the expression level of said target is reduced expression of said target. In still other embodiments, the expression level of said target is increased expression of said target. In some embodiments, the level of expression of said target is determined by using a method selected from the group consisting of in situ hybridization, a PCR-based method, an array-based method, an immunohistochemical method, an RNA assay method and an immunoassay method. In other embodiments, said reagent is selected from the group consisting of a nucleic acid probe, one or more nucleic acid primers, and an antibody. In yet other embodiments, said nucleic acid probe is a reporter probe and/or a capture probe. In still other embodiments, the biological sample is a biopsy. In other embodiments, the biological sample is a urine sample, a blood sample or a prostate tumor sample. In some embodiments, the blood sample is plasma, serum, or whole blood. In still other embodiments, the level of expression is increased or reduced compared to a control. In yet other embodiments, said measuring the level of expression comprises measuring the level of an RNA transcript.

In embodiments of the method, kit or systems disclosed herein, the plurality of targets comprises at least about 2, at least about 3, at least about 4, at least about 5, at least about 6, at least about 7, at least about 8, at least about 9, or at least about 10 targets selected from Table 2. In embodiments of the method, kit or systems disclosed herein, the plurality of targets does not include one or more of the targets listed in Table 2. In embodiments of the method, kit or systems disclosed herein, the plurality of targets includes not more than 200, 175, 150, 125, 100, 75, 50 or 25 of the targets listed in Table 2.

Embodiments of the disclosure include the following non-limiting exemplary numbered embodiments:

1. A method comprising: optionally providing, obtaining, or having obtained a biological sample from a prostate cancer subject; performing or having performed an assay to detect or determine the presence and/or expression level of at least one or more genes selected from Table 2 in a sample from a prostate cancer subject; and administering a treatment to the subject, wherein the treatment is selected from the group consisting of surgery, chemotherapy, radiation therapy, immunotherapy/biological therapy, hormonal therapy, and photodynamic therapy based at least in part on the presence and/or expression level of the at least one or more genes selected from Table 2. 2. The method of any one of the preceding embodiments, wherein the expression level of said target is reduced expression of said target. 3. The method of any one of the preceding embodiments, wherein the expression level of said target is increased expression of said target. 4. The method of any one of the preceding embodiments, wherein the level of expression of said target is detected or determined by using a method selected from the group consisting of in situ hybridization, a PCR-based method, an array-based method, an immunohistochemical method, an RNA assay method and an immunoassay method. 5. The method of any one of the preceding embodiments, wherein said method comprises using a reagent selected from the group consisting of a nucleic acid probe, one or more nucleic acid primers, and an antibody. 6. The method of any one of the preceding embodiments, wherein said nucleic acid probe is a reporter probe and/or a capture probe. 7. A method comprising: (a) optionally providing, obtaining, or having obtained a biological sample from a subject with prostate cancer; (b) performing or having performed an assay to detect or determine the presence and/or expression level in the biological sample for a plurality of targets, wherein the plurality of targets comprises one or more genes selected from Table 2; (c) subtyping or obtaining the subtype of the prostate cancer in the subject based on the presence or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy based at least in part on the subtype of the prostate cancer. 8. The method of any one of the preceding embodiments, wherein the expression level of said target is reduced expression of said target. 9. The method of any one of the preceding embodiments, wherein the expression level of said target is increased expression of said target. 10. The method of any one of the preceding embodiments, wherein the level of expression of said target is detected or determined by using a method selected from the group consisting of in situ hybridization, a PCR-based method, an array-based method, an immunohistochemical method, an RNA assay method and an immunoassay method. 11. The method of any one of the preceding embodiments, wherein said method comprises using a reagent selected from the group consisting of a nucleic acid probe, one or more nucleic acid primers, and an antibody. 12. The method of any one of the preceding embodiments, wherein the target comprises a nucleic acid sequence. 13. The method of any one of the preceding embodiments, wherein the prostate cancer subtype is selected from the group consisting of Luminal Differentiated (LD), Luminal Proliferating (LP), Basal Immune (BI), and Basal Neuroendocrine-like (BN). 14. A system for analyzing a cancer, comprising: (a) A probe set comprising a plurality of target sequences, wherein (i) the plurality of target sequences hybridizes to one or more genes selected from Table 2; or (ii) the plurality of target sequences comprises one or more genes selected from Table 2; and (b) a computer model or algorithm for analyzing an expression level and/or expression profile of the target hybridized to the probe in a sample from a subject suffering from prostate cancer. 15. The system of any one of the preceding embodiments, further comprising a label that specifically binds to the target, the probe, or a combination thereof. 16. A method of treating a subject with prostate cancer, comprising: optionally providing, obtaining, or having obtained a biological sample comprising prostate cancer cells from the subject; performing or having performed an assay to determine or detect the presence and/or level of expression of at least one or more targets selected from Table 2 using at least one reagent that specifically binds to said targets; subtyping or obtaining the subtype of the prostate cancer based on the presence and/or level of expression of the at least one or more targets; and prescribing and/or administering a treatment regimen to the subject based at least in part on the prostate cancer subtype. 17. The method of any one of the preceding embodiments, wherein the prostate cancer subtype is selected from the group consisting of Luminal Differentiated (LD), Luminal Proliferating (LP), Basal Immune (BI), and Basal Neuroendocrine-like (BN). 18. The method of any one of the preceding embodiments, wherein said reagent is selected from the group consisting of a nucleic acid probe, one or more nucleic acid primers, and an antibody. 19. The method of any one of the preceding embodiments, wherein said nucleic acid probe is a reporter probe and/or a capture probe. 20. The method of any one of the preceding embodiments, wherein the treatment regimen is surgery, chemotherapy, radiation therapy, immunotherapy/biological therapy, hormonal therapy, and/or photodynamic therapy. 21. The method of any one of the preceding embodiments, wherein the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, or celecoxicib. 22. A kit for analyzing a prostate cancer, comprising: (a) a probe set comprising a plurality of target sequences, wherein the plurality of target sequences comprises at least one target sequence listed in Table 2; and (b) a computer model or algorithm for analyzing an expression level and/or expression profile of the target sequences in a sample. 23. The kit of any one of the preceding embodiments, further comprising a computer model or algorithm for correlating the expression level or expression profile with disease state or outcome. 24. The kit of any one of the preceding embodiments, further comprising a computer model or algorithm for designating a treatment modality for the individual. 25. The kit of any one of the preceding embodiments, further comprising a computer model or algorithm for normalizing expression level or expression profile of the target sequences. 26. A method comprising: a) optionally providing, obtaining, or having obtained a biological sample from a subject having prostate cancer; b) measuring or obtaining a measure of the levels of expression in the biological sample of a plurality of genes selected from Table 2; and c) subtyping the prostate cancer of the subject according to a genomic subtyping classifier based on the levels of expression of the plurality of genes, wherein said subtyping comprises assigning the prostate cancer to one of four subtypes selected from the group consisting of Luminal Differentiated (LD), Luminal Proliferating (LP), Basal Immune (BI), and Basal Neuroendocrine-like (BN). 27. The method of any one of the preceding embodiments, further comprising administering a treatment to the subject, wherein the treatment is selected from the group consisting of surgery, chemotherapy, radiation therapy, immunotherapy/biological therapy, hormonal therapy, and photodynamic therapy based at least in part on the subtype of the cancer. 28. The method of any one of the preceding embodiments, wherein the expression level of said target is reduced expression of said target. 29. The method of any one of the preceding embodiments, wherein the expression level of said target is increased expression of said target. 30. The method of any one of the preceding embodiments, wherein the level of expression of said target is determined by using a method selected from the group consisting of in situ hybridization, a PCR-based method, an array-based method, an immunohistochemical method, an RNA assay method and an immunoassay method. 31. The method of any one of the preceding embodiments, wherein said reagent is selected from the group consisting of a nucleic acid probe, one or more nucleic acid primers, and an antibody. 32. The method of any one of the preceding embodiments, wherein said nucleic acid probe is a reporter probe and/or a capture probe. 33. The method of any one of the preceding embodiments, wherein the sample is a biopsy. 34. The method of embodiment 33, wherein the biological sample is a urine sample, a blood sample or a prostate tumor sample. 35. The method of embodiment 34, wherein the blood sample is plasma, serum, or whole blood. 36. The method of any one of the preceding embodiments, wherein the subject is a human. 37. The method of any one of the preceding embodiments, wherein the level of expression is increased or reduced compared to a control. 38. The method of any one of the preceding embodiments, wherein said measuring the level of expression comprises measuring the level of an RNA transcript. 39. The method, kit or system of any one of the preceding embodiments, wherein the plurality of targets comprises at least about 2, at least about 3, at least about 4, at least about 5, at least about 6, at least about 7, at least about 8, at least about 9, or at least about 10 targets selected from Table 2. 40. The method, kit or system of any one of the preceding embodiments, wherein the plurality of targets does not include one or more of the targets listed in Table 2. 41. The method, kit or system of any one of the preceding embodiments, wherein the plurality of targets includes not more than 200, 175, 150, 125, 100, 75, 50 or 25 of the targets listed in Table 2.

Embodiments of the disclosure include the following non-limiting exemplary numbered embodiments:

1. A method comprising: (a) optionally providing, obtaining, or having obtained a biological sample from a prostate cancer subject; (b) performing or having performed an assay to detect or determine the presence and/or expression level of at least one or more target genes selected from Table 2 in a sample from a prostate cancer subject; and (c) administering a treatment to the subject, wherein the treatment is selected from the group consisting of surgery, chemotherapy, radiation therapy, immunotherapy/biological therapy, hormonal therapy, and photodynamic therapy based at least in part on the presence and/or expression level of the at least one or more genes selected from Table 2.

2. A method comprising: (a) optionally providing, obtaining, or having obtained a biological sample from a subject with prostate cancer; (b) performing or having performed an assay to detect or determine the presence and/or expression level in the biological sample for a plurality of targets, wherein the plurality of targets comprises one or more genes selected from Table 2; (c) subtyping or obtaining the subtype of the prostate cancer in the subject based on the presence or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy based at least in part on the subtype of the prostate cancer.

3. The method of embodiment 2, wherein the prostate cancer subtype is selected from the group comprising or consisting of Luminal Differentiated (LD), Luminal Proliferating (LP), Basal Immune (BI), and Basal Neuroendocrine-like (BN).

4. A method comprising: (a) optionally providing, obtaining, or having obtained a biological sample from a subject having prostate cancer; (b) measuring or obtaining a measure of the levels of expression in the biological sample of a plurality of target genes selected from Table 2; and (c) subtyping the prostate cancer of the subject according to a genomic subtyping classifier based on the levels of expression of the plurality of target genes, wherein said subtyping comprises assigning the prostate cancer to a subtype selected from the group comprising or consisting of Luminal Differentiated (LD), Luminal Proliferating (LP), Basal Immune (BI), and Basal Neuroendocrine-like (BN).

5. The method of embodiment 4, further comprising prescribing a treatment regimen to the subject based at least in part on the prostate cancer subtype.

6. The method of embodiment 4 or 5, further comprising administering a treatment to the subject, based at least in part on the subtype of the cancer.

7. The method of any one of embodiments 4-6, wherein the treatment is selected from the group consisting of surgery, chemotherapy, radiation therapy, immunotherapy/biological therapy, hormonal therapy, and photodynamic therapy.

8. The method of any one of embodiments 1-7, wherein the expression level of said target is reduced expression of said target.

9. The method of any one of embodiments 1-7, wherein the expression level of said target is increased expression of said target.

10. The method of any one of embodiments 1-9, wherein the level of expression of said target is detected or determined by using a method selected from the group consisting of in situ hybridization, a PCR-based method, an array-based method, an immunohistochemical method, an RNA assay method and an immunoassay method.

11. The method of any one of embodiments 1-10, wherein said method comprises using a reagent selected from the group consisting of a nucleic acid probe, one or more nucleic acid primers, and an antibody.

12. The method of embodiment 11, wherein said nucleic acid probe is a reporter probe and/or a capture probe.

13. A method of treating a subject with prostate cancer, comprising: (a) optionally providing, obtaining, or having obtained a biological sample comprising prostate cancer cells from the subject; (b) performing or having performed an assay to determine or detect the presence and/or level of expression of at least one or more targets selected from Table 2 using at least one reagent that specifically binds to said targets; (c) subtyping or obtaining the subtype of the prostate cancer based on the presence and/or level of expression of the at least one or more targets; and (d) prescribing and/or administering a treatment regimen to the subject based at least in part on the prostate cancer subtype.

14. The method of embodiment 13, wherein the prostate cancer subtype is selected from the group consisting of Luminal Differentiated (LD), Luminal Proliferating (LP), Basal Immune (BI), and Basal Neuroendocrine-like (BN).

15. The method of any one of embodiments 13-14, wherein said reagent is selected from the group consisting of a nucleic acid probe, one or more nucleic acid primers, and an antibody.

16. The method of embodiment 15, wherein said nucleic acid probe is a reporter probe and/or a capture probe.

17. The method of any one of embodiments 13-16, wherein the treatment regimen is surgery, chemotherapy, radiation therapy, immunotherapy/biological therapy, hormonal therapy, and/or photodynamic therapy.

18. The method of any one of embodiments 1-17, wherein the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, or celecoxicib.

19. The method of any one of embodiments 1-18, comprising subtyping the subject as having LD prostate cancer.

20. The method of embodiment 19, further comprising predicting the subject as benefiting from radiotherapy and/or radical prostatectomy, optionally benefiting more than a subject having LP, BI or BN prostate cancer.

21. The method of embodiment 19 or 20, further comprising prescribing and/or administering primary radiotherapy and/or radical prostatectomy.

22. The method of embodiment 19, further comprising predicting the subject as not benefiting from docetaxel in addition to ADT.

23. The method of embodiment 19 or 22, further comprising not prescribing and/or not administering docetaxel in addition to ADT.

24. The method of embodiment 19, further comprising characterizing the LD prostate cancer as one or more of: androgen receptor (AR) driven; having high expression levels of prostate terminal differentiation markers, optionally higher than LP, BI or BN prostate cancer; having lower metastatic potential, optionally wherein the metastatic potential is lower than LP and BI prostate cancer; and/or being sensitive to ADT.

25. The method of embodiment 19 or 24, further comprising prescribing and/or administering ADT.

26. The method of any one of embodiments 1-18, comprising subtyping the subject as having LP prostate cancer.

27. The method of embodiment 26, further comprising predicting the subject as benefiting from: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel.

28. The method of embodiment 26 or 29, further comprising prescribing and/or administering: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel.

29. The method of embodiment 26, further comprising predicting the subject as benefiting from docetaxel in addition to ADT.

30. The method of embodiment 26 or 29, further comprising prescribing and/or administering docetaxel in addition to ADT.

31. The method of embodiment 26, further comprising characterizing the LP prostate cancer as one or more of: androgen receptor (AR) driven; having high expression levels of proliferation markers, optionally higher than LD, BI and BN prostate cancer; having higher metastatic potential, optionally higher than LD prostate cancer; insensitive to ADT; and/or sensitive to taxane-based chemotherapy and androgen receptor signaling inhibitors (ARSI).

32. The method of embodiment 26 or 31, further comprising not prescribing and/or not administering ADT.

33. The method of embodiment 26 or 31, further comprising prescribing and/or administering a taxane-based chemotherapy and/or an ARSI.

34. The method of embodiment 26, further comprising predicting the subject as benefiting less from primary radiotherapy and/or radical prostatectomy than a subject with LD prostate cancer.

35. The method of embodiment 26, further comprising predicting the subject as benefiting more from primary radiotherapy than a subject with BN prostate cancer.

36. The method of any one of the preceding embodiments, comprising subtyping the subject as having LD prostate cancer, or, comprising subtyping the subject as having LP prostate cancer.

37. The method of embodiment 36, further comprising predicting the subject as not benefiting from higher dose primary radiotherapy, optionally wherein the higher dose primary radiotherapy is 79 Gy.

38. The method of embodiment 36 or 37, further comprising not prescribing and/or not administering a higher dose primary radiotherapy, optionally wherein the higher dose primary radiotherapy is 79 Gy.

39. The method of embodiment 36, 37 or 38, further comprising prescribing and/or administering a lower dose primary radiotherapy, optionally wherein the lower dose primary radiotherapy is 70 Gy.

40. The method of embodiment 36, further comprising predicting the subject as not benefitting from long term adjuvant ADT, optionally wherein the long term ADT is 28 months.

41. The method of embodiment 36 or 40, further comprising not prescribing and/or not administering long term adjuvant ADT, optionally wherein the long term ADT is 28 months.

42. The method of embodiment 36, 40 or 41, further comprising prescribing and/or administering short term adjuvant ADT, optionally wherein the short term ADT is 4 months.

43. The method of embodiment 36, further comprising predicting the subject as benefiting from the addition of long term ADT to salvage RT in combination with following biochemical recurrence, optionally wherein long term ADT is 24 months.

44. The method of embodiment 36 or 43, further comprising prescribing and/or administering long term ADT in addition to salvage RT following biochemical recurrence, optionally wherein long term ADT is 24 months.

45. The method of embodiment 36, further comprising predicting the subject as benefiting from abiraterone acetate.

46. The method of embodiment 36 or 45, further comprising prescribing and/or administering abiraterone acetate.

47. The method of any one of embodiments 1-18, comprising subtyping the subject as having BN prostate cancer.

48. The method of embodiment 47, further comprising predicting the subject as benefiting from: an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib.

49. The method of embodiment 47 or 48, further comprising prescribing and/or administering: an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib.

50. The method of embodiment 47, further comprising predicting the subject as not benefiting from docetaxel in addition to ADT.

51. The method of embodiment 47 or 50, further comprising not prescribing and/or not administering docetaxel in addition to ADT.

52. The method of embodiment 47, further comprising predicting the subject as not benefiting from abiraterone acetate.

53. The method of embodiment 47 or 52, further comprising not prescribing and/or not administering abiraterone acetate.

54. The method of embodiment 47, further comprising predicting the subject as benefiting less from primary radiotherapy and/or radical prostatectomy than a subject with LD prostate cancer.

55. The method of embodiment 47, further comprising predicting the subject as benefiting less from primary radiotherapy than a subject with LD, BI or LP prostate cancer.

56. The method of embodiment 47, further comprising characterizing the BN prostate cancer as one or more of: non-AR driven; having the low expression of prostate terminal differentiation markers, optionally lower than LD, LP and BI prostate cancer; high expression of markers of a suppressed tumor immune microenvironment, optionally higher than LD, LP and BI prostate cancer; being resistant to ADT; and/or being sensitive to platinum and vinca alkaloid chemotherapies.

57. The method of embodiment 47 or 56, further comprising prescribing and/or administering a platinum and/or a vinca alkaloid chemotherapy.

58. The method of embodiment 47 or 56, further comprising not prescribing and/or not administering ADT.

59. The method of any one of embodiments 1-18, comprising subtyping the subject as having BI prostate cancer.

60. The method of embodiment 59, further comprising predicting the subject as benefiting from: a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin.

61. The method of embodiment 59 or 60, further comprising prescribing and/or administering: a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin.

62. The method of embodiment 59, further comprising predicting the subject as not benefiting from docetaxel in addition to ADT.

63. The method of embodiment 59 or 62, further comprising not prescribing and/or not administering docetaxel in addition to ADT.

64. The method of embodiment 59, further comprising predicting the subject as benefiting less from primary radiotherapy and/or radical prostatectomy than a subject with LD prostate cancer.

65. The method of embodiment 59, further comprising predicting the subject as benefiting more from primary radiotherapy than a subject with BN prostate cancer.

66. The method of embodiment 59, further comprising characterizing the BI prostate cancer as one or more of: non-AR driven; having elevated expression of other sex steroid transcription factors, optionally estrogen receptor, glucocorticoid receptor and progesterone receptors; sensitive to ADT; having high expression of markers of an activated tumor immune microenvironment, optionally higher than LD, LP and BN prostate cancer; having higher metastatic potential, optionally higher than LD prostate cancer; and/or sensitive to radiotherapy, protein kinase inhibitors and immune-checkpoint therapy.

67. The method of embodiment 59 or 66, further comprising prescribing and/or administering ADT, radiotherapy, a protein kinase inhibitor and/or immune-checkpoint therapy.

68. The method of any one of 1-18, or 47-67, comprising subtyping the subject as having BN prostate cancer, or, comprising subtyping the subject as having BI prostate cancer.

69. The method of embodiment 68, further comprising predicting the subject as benefiting from higher dose primary radiotherapy, optionally wherein the higher dose primary radiotherapy is 79 Gy.

70. The method of embodiment 68 or 69, further comprising prescribing and/or administering a higher dose primary radiotherapy, optionally wherein the higher dose primary radiotherapy is 79 Gy.

71. The method of embodiment 68, 69 or 70, further comprising not prescribing and/or not administering a lower dose primary radiotherapy, optionally wherein the lower dose primary radiotherapy is 70 Gy.

72. The method of embodiment 68, further comprising predicting the subject as benefiting from long term adjuvant ADT, optionally wherein the long term ADT is 28 months.

73. The method of embodiment 68 or 72, further comprising prescribing and/or administering long term adjuvant ADT, optionally wherein the long term ADT is 28 months.

74. The method of embodiment 68, 72 or 73, further comprising not prescribing and/or not administering short term adjuvant ADT, optionally wherein the short term ADT is 4 months.

75. The method of embodiment 68, further comprising predicting the subject as not benefiting from the addition of long term ADT to salvage RT in combination with following biochemical recurrence, optionally wherein long term ADT is 24 months.

76. The method of embodiment 68 or 75, further comprising not prescribing and/or administering long term ADT in addition to salvage RT following biochemical recurrence, optionally wherein long term ADT is 24 months.

77. The method of any one of the preceding embodiments, wherein the sample or biological sample is a biopsy, urine sample, a blood sample or a prostate tumor sample.

78. The method of embodiment 77, wherein the blood sample is plasma, serum, or whole blood.

79. The method of any one of the preceding embodiments, wherein the subject is a human.

80. The method of any one of the preceding embodiments, wherein the level of expression is increased or reduced compared to a control.

81. The method of any one of the preceding embodiments, wherein said measuring the level of expression comprises measuring the level of an RNA transcript.

82. The method of any one of the preceding embodiments, wherein the plurality of targets are nucleic acid targets.

83. The method of embodiment 82, wherein the plurality of nucleic acid targets comprises a coding target.

84. The method of embodiment 83, wherein the coding target is an exonic sequence.

85. The method of any one of embodiments 82-84, wherein the plurality of nucleic acid targets comprises a non-coding target.

86. The method of embodiment 85 wherein the non-coding target comprises an intronic sequence or partially overlaps an intronic sequence.

87. The method of embodiment 85, wherein the non-coding target comprises an intergenic sequence.

88. The method of embodiment 85, wherein the non-coding target comprises a sequence within the untranslated region (UTR) or partially overlaps with a UTR sequence.

89. The method of any one of embodiments 82-88, wherein the plurality of nucleic acid targets comprise a DNA sequence.

90. The method of any one of embodiments 82-88, wherein the plurality of nucleic acid targets comprise an RNA sequence.

91. The method of any one of embodiments 82-90, further comprising sequencing the plurality of nucleic acid targets.

92. The method of any one of embodiments 82-90, further comprising hybridizing the plurality of nucleic acid targets to a solid support.

93. The method of embodiment 92, wherein the solid support is a bead or array.

94. A probe set for use in the method of any one of the preceding embodiments, the probe set comprising or consisting of a plurality of probes, wherein the probes in the set are used for detecting the expression level of a plurality of nucleic acid targets in a sample from the subject, wherein the plurality of nucleic acid targets comprise or consist of a plurality of targets selected the targets in Table 2, optionally wherein the probes of the probe set hybridize to the nucleic acid targets.

95. A probe set for subtyping, prognosing and/or predicting benefit from prostate cancer therapy of a prostate cancer in a subject comprising or consisting of a plurality of probes, wherein the probes in the set are used for detecting the expression level of a plurality of nucleic acid targets in a sample from the subject, wherein the plurality of nucleic acid targets comprise or consist of a plurality of targets selected the targets in Table 2, optionally wherein the probes of the probe set hybridize to the nucleic acid targets.

96. The probe set of embodiment 94 or 95, wherein the plurality of nucleic acid targets comprises a coding target.

97. The probe set of embodiment 96, wherein the coding target is an exonic sequence.

98. The probe set of any one of embodiments 94-97, wherein the plurality of nucleic acid targets comprises a non-coding target.

99. The probe set of embodiment 98, wherein the non-coding target comprises an intronic sequence or partially overlaps an intronic sequence.

100. The probe set of embodiment 98, wherein the non-coding target comprises an intergenic sequence.

101. The probe set of embodiment 98, wherein the non-coding target comprises a sequence within the untranslated region (UTR) or partially overlaps with a UTR sequence.

102. The probe set of any one of embodiments 94-101, wherein the plurality of nucleic acid targets comprise a DNA sequence.

103. The probe set of any one of embodiments 94-101, wherein the plurality of nucleic acid targets comprise an RNA sequence.

104. A system for analyzing a cancer, comprising:
(a) the probe set of any one of embodiments 94-103, and
(b) a computer model or algorithm for analyzing an expression level and/or expression profile of the target hybridized to the probe in a sample from a subject suffering from prostate cancer.

105. The system of embodiment 104, further comprising a computer model or algorithm for correlating the expression level or expression profile with disease state or outcome.

106. The system of embodiment 104, further comprising a computer model or algorithm for designating a treatment modality for the subject.

107. The system of embodiment 104, further comprising a computer model or algorithm for normalizing expression level or expression profile of the target sequences.

108. The method, probe set, or system of any one of the preceding embodiments, wherein the plurality of targets comprises at least about 2, at least about 3, at least about 4, at least about 5, at least about 6, at least about 7, at least about 8, at least about 9, or at least about 10 targets selected from Table 2.

109. The method, probe set, or system of any one of the preceding embodiments, wherein the plurality of targets comprises or consists of at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 targets selected from the targets listed in Table 2, 2.1, 2.2, 2.3, 2.4, 2.5, 2.6, 2.7, 2.8, 2.9, 2.10, or 2.11.

110. The method, probe set, or system of any one of the preceding embodiments, wherein the plurality of targets does not include one or more of the targets listed in Table 2.

111. The method, probe set, or system of any one of the preceding embodiments, wherein the plurality of targets includes not more than 215, 210, 200, 175, 150, 125, 110, 100, 90, 80, 70, 60, 50, 40, 30, 20, 15, 14, 13, 12, 11, 10, 9, 8, 7, 6, or 5 of the targets listed in Table 2, 2.1, 2.2, 2.3, 2.4, 2.5, 2.6, 2.7, 2.8, 2.9, 2.10, or 2.11.

112. The method, probe set, or system of any one of the preceding embodiments, wherein the plurality of targets comprises or consists of targets having an absolute value of the coefficient value in Table 2 of at least 0.10000, 0.12500, 0.15000, 0.17500, 0.20000, 0.22500, 0.25000, 0.27500, 0.30000, 0.32500, 0.35000, 0.37500, 0.40000, 0.42500, 0.45000, 0.47500, 0.50000, 0.52500, 0.55000, 0.57500, 0.60000, 0.62500, 0.65000, 0.67500, 0.70000, 0.72500, 0.75000, 0.77500, 0.80000, 0.82500, 0.85000, 0.87500, 0.90000, 0.92500, 0.95000, 0.97500, 1.00000, 1.50000, 2.00000, 2.50000, 3.00000, 3.50000, or 4.00000.

It is further described an in vitro method for determining a prostate cancer subtype, for predicting clinical outcome of a subject with prostate cancer, or for predicting a response to treatment, which method comprises detecting or determining the presence and/or expression level of at least one or more target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample from a prostate cancer subject. The biological sample is preferably a biopsy or prostate tumor sample.

The level of expression of said target may be determined e.g. by in situ hybridization, a PCR-based method, an array-based method, an immunohistochemical method, an RNA assay method and an immunoassay method, most preferably a PCR-based method. In a preferred embodiment, the method makes use of a computer model or algorithm for analyzing an expression level and/or expression profile of the target.

It is more particularly described an in vitro method for subtyping a prostate cancer in a subject, which method comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, whereby the prostate cancer is assigned to a subtype that is Luminal Differentiated (LD), Luminal Proliferating (LP), Basal Immune (BI), or Basal Neuroendocrine-like (BN) subtype.

It is also provided an in vitro method for assessing whether a subject with prostate cancer is likely to benefit from radiotherapy and/or radical prostatectomy, which method comprises identifying the prostate cancer as an LD subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with an LD subtype is likely to benefit from radiotherapy and/or radical prostatectomy.

It is also provided an in vitro method for assessing whether a subject with prostate cancer is likely to benefit from docetaxel in addition to ADT, which method comprises identifying the prostate cancer as an LD, LP, BN or BI subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with an LD, BN or BI subtype is not likely to benefit from doxetaxel in addition to ADT, while a subject with an LP subtype is likely to benefit from doxetaxel in addition to ADT.

It is also provided an in vitro method for assessing whether a subject with prostate cancer is likely to benefit a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel, which method comprises identifying the prostate cancer as a LP subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a LP subtype is likely to benefit from said drugs.

In particular a subject with LP prostate cancer is predicted as benefiting less from primary radiotherapy and/or radical prostatectomy than a subject with LD prostate cancer, but also as benefiting more from primary radiotherapy than a subject with BN prostate cancer.

In particular a subject with BI prostate cancer is predicted as benefiting less from primary radiotherapy and/or radical prostatectomy than a subject with LD prostate cancer, but also as benefiting more from primary radiotherapy than a subject with BN prostate cancer.

It is thus also provided an in vitro method for determining whether a subject with prostate cancer is likely to benefit from a higher dose primary radiotherapy, optionally wherein the higher dose primary radiotherapy is 79 Gy, which method comprises identifying the prostate cancer as a LP or LD subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a LP or LD subtype is not likely to benefit from said higher dose primary radiotherapy.

It is thus also provided an in vitro method for determining whether a subject with prostate cancer is likely to benefit from a long term adjuvant ADT, optionally wherein the long term ADT is 28 months, which method comprises identifying the prostate cancer as a LP, LD or BI subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a LP or LD subtype is not likely to benefit from said long term adjuvant ADT, while a subject with a BI subtype is likely to benefit from said long term adjuvant ADT.

In particular a subject with BI subtype will likely not benefit from the addition of long term ADT to salvage RT in combination with following biochemical recurrence, optionally wherein long term ADT is 24 months.

It is thus also provided an in vitro method for determining whether a subject with prostate cancer is likely to benefit from abiraterone acetate, which method comprises identifying the prostate cancer as a LP, LD or BN subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a LP, or LD subtype is likely to benefit from abiraterone acetate, while a subject with BN subtype is not likely to benefit from abiraterone acetate.

It is further provided an in vitro method for determining whether a subject with prostate cancer is likely to benefit from an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib, which method comprises identifying the prostate cancer as a BN subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a BN subtype is likely to benefit from said drugs.

It is thus also provided an in vitro method for determining whether a subject with prostate cancer is likely to benefit from a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin, which method comprises identifying the prostate cancer as a BI subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a BI subtype is likely to benefit from said drugs.

It is thus also provided an in vitro method for determining whether a subject with prostate cancer is likely to benefit from a higher dose primary radiotherapy, optionally wherein the higher dose primary radiotherapy is 79 Gy, which method comprises identifying the prostate cancer as a BN or BI subtype by means of the above method which comprises measuring or obtaining a measure of the levels of expression of a plurality of target genes selected from Table 2, optionally at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 target genes selected from the genes listed in Table 2, 2.1, 2.2, 2.3, 2.4, 2.5, 2.6, 2.7, 2.8, 2.9, 2.10, or 2.11, in a biological sample of the subject, wherein a subject with a BN or BI subtype is likely to benefit from said higher dose primary radiotherapy.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 sets forth an embodiment of data showing microarray expression data for the 21 seeding features used in the PSC model to cluster patient samples in the training and testing cohorts (n=64,000).

FIG. 2 sets forth an embodiment of data showing molecular subtypes proportions across NCCN risk groups.

FIGS. 3A-3B set forth an embodiment of data showing subtype prognosis after primary radiation therapy (A) and radical prostatectomy (B).

FIGS. 4A-4B set forth an embodiment of data showing outcomes for subtypes with dose-escalated radiotherapy.

FIGS. 5A-5B set forth an embodiment of data showing outcomes for subtypes with long term (LT) androgen deprivation therapy (ADT) as compared to short term (ST) ADT.

FIGS. 6A-6B set forth an embodiment of outcomes for subtypes with salvage radiation (RT) plus ADT as compared to RT alone.

FIGS. 7A-7B set forth an embodiment of data showing outcomes for subtypes with docetaxel in addition to ADT.

### DETAILED DESCRIPTION

The present disclosure provides systems and methods for diagnosing, prognosing, treating, and/or monitoring the status or outcome of a prostate cancer in a subject using expression-based analysis of a plurality of targets. Generally, the method comprises (a) optionally providing, obtaining, or having obtained, a sample from a subject; (b) performing or having performed an assay of the expression level for a plurality of targets in the sample; and (c) diagnosing, prognosing, treating, and/or monitoring the status or outcome of a prostate cancer based on the expression level of the plurality of targets.

Performing or having performed an assay of the expression level for a plurality of targets in the sample may comprise applying the sample to a microarray. In some instances, performing or having performed an assay of the expression level may comprise the use of an algorithm. The algorithm may be used to produce a classifier. Alternatively, the classifier may comprise a probe selection region. In some instances, performing or having performed an assay of the expression level for a plurality of targets comprises detecting and/or quantifying the plurality of targets. In some embodiments, performing or having performed an assay of the expression level for a plurality of targets comprises sequencing the plurality of targets. In some embodiments, performing or having performed an assay of the expression level for a plurality of targets comprises amplifying the plurality of targets. In some embodiments, performing or having performed an assay of the expression level for a plurality of targets comprises quantifying the plurality of targets. In some embodiments, performing or having performed an assay of the expression level for a plurality of targets comprises conducting a multiplexed reaction on the plurality of targets. In other embodiments, performing or having performed an assay of the expression level for a plurality of targets comprises quantifying the targets using one or more reporter probes and one or more capture probes. In some embodiments, the expression level for a plurality of targets in the sample is obtained from a report or other source of information which includes the results of an assay described herein.

In some instances, the plurality of targets comprises one or more targets selected from Table 2. In some instances, the plurality of targets comprises at least about 2, at least about 3, at least about 4, at least about 5, at least about 6, at least about 7, at least about 8, at least about 9, or at least about 10 targets selected from Table 2. In some embodiments, the plurality of targets does not include one or more of the targets listed in Table 2. In some embodiments, the plurality of targets includes not more than 200, 175, 150, 125, 100, 75, 50 or 25 of the targets listed in Table 2.

Further disclosed herein are methods for subtyping prostate cancer. Generally, the method comprises: (a) optionally providing, obtaining, or having obtained a sample comprising prostate cancer cells from a subject; (b) performing or having performed an assay of the expression level for a plurality of targets in the sample; and (c) subtyping the cancer based on the expression level of the plurality of targets. In some instances, the plurality of targets comprises one or more targets selected from Table 2. In some instances, the plurality of targets comprises at least about 2, at least about 3, at least about 4, at least about 5, at least about 6, at least about 7, at least about 8, at least about 9, or at least about 10 targets selected from Table 2. In some embodiments, the plurality of targets does not include one or more of the targets listed in Table 2. In some embodiments, the plurality of targets includes not more than 200, 175, 150, 125, 100, 75, 50 or 25 of the targets listed in Table 2. In some embodiments, the plurality of targets is ABCC11, ACOXL, ACTC1, AKAP2, AMACR, AMD1, ANXA8, AP2S1, APELA, APLN, AQP9, ARHGEF1, ATP1B2, B3GNT5, BCHE, BTN3A1, BTN3A3, C12orf75, CIS, CACNA1D, CAMK2N1, CAMK4, CBS, CCDC169, CD200, CD37, CD3D, CD70, CDH11, CDH19, CDH7, CDO1, CENPF, CFB, CGN, CHD3, CHRM3, CHST9, CKM, CLDN10, CLDN4, CLSTN2, COL1A1, COL3A1, COL4A2, COL8A1, CP, CPA6, CPLX3, CSGALNACT1, CSTA, CTTN, CYP4F11, CYP4F2, CYTH2, DDX21, DPT, EDN3, E124, EIF3B, FABP4, FAM111B, FAM153A, FAM155A, FAP, FAR2P1, FILIP1L, FLNC, GABRD, GATA3, GBP1, GCNT1, GFRA3, GJB2, GOLGA8A, GPR158, GPR171, GPT2, GTF2I, HDAC9, HIST1H1E, HIST1H2AD, HJURP, HLA-DMB, HLA-F, HLF, HMOX1, HOXA-AS3, HS6ST2, IKZF1, IKZF3, IRF1, ITGB6, ITGBL1, ITPR1, KHDRBS3, KL, KLHL5, KLK12, KRT14, LAMP5, LGR5, LINC00668, LRG1, LRRC9, LY6K, MAOB, MCM7, MEIS1, MEX3A, MFAP5, MKRN1, MORC2, MUC1, MUC12, MUC15, MUC16, MUC2, MYL2, MYOC, MYOM1, NPM1, NPY4R, NRK, ODC1, ONECUT2, OPTN, OR2L13, OR51F1, OR52N5, ORM1, OS9, OXCT1, P2RY14, PABPC3, PARG, PCDHA12, PDAP1, PDE11A, PICK1, PIGR, PLCXD3, PLEK, PMM2, PNMA1, PODXL2, POTEB3, POTEC, POTED, PPP1R14B, PPP1R18, PRKCB, PRKG2, PYHIN1, RAP1A, REXO2, RIMS1, RLIM, RND3, RNPS1, RPAIN, RPL14, RPL22, RPS3, RRM2, RSPO3, S100A2, S100A9, S100P, SCUBE2, SERPINB5, SERTM1, SET, SKA3, SLC39A7, SLC44A5, SLC5A4, SLFN11, SMS, SNAP91, SPINK1, SRPX2, SSRP1, STAC, STAU2-AS1, SUPT5H, SYCE1, SYT6, TBC1D3B, TBX4, THBS4, TLR1, TNNI1, TPM2, TSEN15, TSPYL2, TUBB4A, TUFM, UGDH, UGT2B4, UPK1A, UQCRH, USP17L10, VCAM1, VSTM2L, VTCN1, VWA2, WDR93, WIFI, WISP1, ZC3H13, ZCCHC13, ZEB2, ZNF556, and/or ZNF608.

In some instances, subtyping the prostate cancer comprises determining whether the cancer would respond to an anti-cancer therapy. Alternatively, subtyping the prostate cancer comprises identifying the cancer as non-responsive to an anti-cancer therapy. Optionally, subtyping the prostate cancer comprises identifying the cancer as responsive to an anti-cancer therapy.

In some embodiments, disclosed is a method comprising: (a) optionally providing, obtaining, or having obtained a biological sample from a prostate cancer subject; (b) performing or having performed an assay to detect or determine the presence and/or expression level of at least one or more target genes selected from Table 2 in a sample from a prostate cancer subject; and (c) administering a treatment to the subject, wherein the treatment is selected from the group consisting of surgery, chemotherapy, radiation therapy, immunotherapy/biological therapy, hormonal therapy, and photodynamic therapy based at least in part on the presence and/or expression level of the at least one or more genes selected from Table 2.

In some embodiments, disclosed is a method comprising: (a) optionally providing, obtaining, or having obtained a biological sample from a subject with prostate cancer; (b) performing or having performed an assay to detect or determine the presence and/or expression level in the biological sample for a plurality of targets, wherein the plurality of targets comprises one or more genes selected from Table 2; (c) subtyping or obtaining the subtype of the prostate cancer in the subject based on the presence or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy based at least in part on the subtype of the prostate cancer. In some embodiments, the prostate cancer subtype is selected from the group comprising or consisting of Luminal Differentiated (LD), Luminal Proliferating (LP), Basal Immune (BI), and Basal Neuroendocrine-like (BN).

In some embodiments, disclosed is a method comprising (a) optionally providing, obtaining, or having obtained a biological sample from a subject having prostate cancer; (b) measuring or obtaining a measure of the levels of expression in the biological sample of a plurality of target genes selected from Table 2; and (c) subtyping the prostate cancer of the subject according to a genomic subtyping classifier based on the levels of expression of the plurality of target genes, wherein said subtyping comprises assigning the prostate cancer to a subtype selected from the group comprising or consisting of Luminal Differentiated (LD), Luminal Proliferating (LP), Basal Immune (BI), and Basal Neuroendocrine-like (BN). In some embodiments, the method further comprises prescribing a treatment regimen to the subject based at least in part on the prostate cancer subtype. In some embodiments, the method further comprises administering a treatment to the subject, based at least in part on the subtype of the cancer. In some embodiments, the treatment is selected from the group consisting of surgery, chemotherapy, radiation therapy, immunotherapy/biological therapy, hormonal therapy, and photodynamic therapy.

In some embodiments disclosed is a method of treating a subject with prostate cancer, comprising: (a) optionally providing, obtaining, or having obtained a biological sample comprising prostate cancer cells from the subject; (b) performing or having performed an assay to determine or detect the presence and/or level of expression of at least one or more targets selected from Table 2 using at least one reagent that specifically binds to said targets; (c) subtyping or obtaining the subtype of the prostate cancer based on the presence and/or level of expression of the at least one or more targets; and (d) prescribing and/or administering a treatment regimen to the subject based at least in part on the prostate cancer subtype.

In some embodiments, the plurality of targets selected from the targets listed in Table 2 comprises or consists of at least 2, 3, 4, 5, 6, 7, 8, 9, 10, 11, 12, 13, 14, 15, or 20 targets, or a range defined by any two of the preceding values (e.g., 2-20, 5-20, 10-20, or 5-10) selected from the subset of 20 targets listed in Table 2.1.

TABLE 2.1

| Number | Accuracy | Targets |
|---|---|---|
| 20 | 85% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L |

In some embodiments, the plurality of targets selected from the targets listed in Table 2 comprises or consists of at least 2, 3, 4, 5, 6, 7, 8, 9, 10, 11, 12, 13, 14, 15, 20, 25, or 30 targets, or a range defined by any two of the preceding values (e.g., 2-30, 5-30, 10-30, 20-30, 10-20 or 5-10) selected from the subset of 30 targets listed in Table 2.2.

TABLE 2.2

| Number | Accuracy | Targets |
|---|---|---|
| 30 | 88% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, or 40 targets, or a range defined by any two of the preceding values (e.g., 2-40, 5-40, 10-40, 20-40, 30-40, 10-20 or 5-10) selected from the subset of 40 targets listed in Table 2.3.

TABLE 2.3

| Number | Accuracy | Targets |
|---|---|---|
| 40 | 90% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, or 50 targets, or a range defined by any two of the preceding values (e.g., 2-50, 5-50, 10-50, 20-50, 40-50, 10-20 or 5-10) selected from the subset of 50 targets listed in Table 2.4.

TABLE 2.4

| Number | Accuracy | Targets |
|---|---|---|
| 50 | 91% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, or 60 targets, or a range defined by any two of the preceding values (e.g., 2-60, 5-60, 10-60, 20-60, 50-60, 10-20 or 5-10) selected from the subset of 60 targets listed in Table 2.5.

TABLE 2.5

| Number | Accuracy | Targets |
|---|---|---|
| 60 | 92% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, or 70 targets, or a range defined by any two of the preceding values (e.g., 2-70, 5-70, 10-70, 20-70, 50-70, 10-20 or 5-10) selected from the subset of 70 targets listed in Table 2.6.

TABLE 2.6

| Number | Accuracy | Targets |
|---|---|---|
| 70 | 93% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1, WISP1, |
| | | CD37, S100A2, TSEN15, CSGALNACT1, LAMP5, VCAM1, |
| | | GPR171, ARHGEF1, POTEC |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, 70, or 80 targets, or a range defined by any two of the preceding values (e.g., 2-80, 5-80, 10-80, 20-80, 50-80, 10-20 or 5-10) selected from the subset of 80 targets listed in Table 2.7.

TABLE 2.7

| Number | Accuracy | Targets |
|---|---|---|
| 80 | 93% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1, WISP1, |
| | | CD37, S100A2, TSEN15, CSGALNACT1, LAMP5, VCAM1, |
| | | GPR171, ARHGEF1, POTEC, CAMK2N1, VWA2, MUC16, SYT6, |
| | | CYP4F11, IKZF1, PPP1R14B, IRF1, CD70, GPT2 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, 70, 80, or 90 targets, or a range defined by any two of the preceding values (e.g., 2-90, 5-90, 10-90, 20-90, 50-90, 10-20 or 5-10) selected from the subset of 90 targets listed in Table 2.8.

TABLE 2.8

| Number | Accuracy | Targets |
|---|---|---|
| 90 | 94% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1, WISP1, |
| | | CD37, S100A2, TSEN15, CSGALNACT1, LAMP5, VCAM1, |
| | | GPR171, ARHGEF1, POTEC, CAMK2N1, VWA2, MUC16, SYT6, |
| | | CYP4F11, IKZF1, PPP1R14B, IRF1, CD70, GPT2, PDAP1, ABCC11, |
| | | CTTN, APLN, KRT14, KLHL5, ITPR1, ZNF608, PRKCB, MCM7 |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, 70, 80, 90, or 100 targets, or a range defined by any two of the preceding values (e.g., 2-100, 5-100, 10-100, 20-100, 50-100, 10-20 or 5-10) selected from the subset of 100 targets listed in Table 2.9.

TABLE 2.9

| Number | Accuracy | Targets |
|---|---|---|
| 100 | 94% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1, WISP1, |
| | | CD37, S100A2, TSEN15, CSGALNACT1, LAMP5, VCAM1, |
| | | GPR171, ARHGEF1, POTEC, CAMK2N1, VWA2, MUC16, SYT6, |
| | | CYP4F11, IKZF1, PPP1R14B, IRF1, CD70, GPT2, PDAP1, ABCC11, |
| | | CTTN, APLN, KRT14, KLHL5, ITPR1, ZNF608, PRKCB, MCM7, |
| | | RLIM, RSPO3, MFAP5, CGN, COL8A1, BCHE, EIF3B, MKRN1, |
| | | FABP4, NPY4R |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, 70, 80, 90, 100, or 110 targets, or a range defined by any two of the preceding values (e.g., 2-110, 5-110, 10-110, 20-110, 50-110, 100-110, 10-20 or 5-10) selected from the subset of 110 targets listed in Table 2.10.

TABLE 2.10

| Number | Accuracy | Targets |
|---|---|---|
| 110 | 95% | GBP1, CENPF, SLC39A7, CD3D, C1S, SSRP1, PICK1, SKA3, |
| | | PPP1R18, ONECUT2, RRM2, B3GNT5, PLEK, EDN3, SMS, RNPS1, |
| | | RIMS1, FAM111B, GATA3, FILIP1L, PYHIN1, ACOXL, GABRD, |
| | | MORC2, OS9, S100A9, CDH11, IKZF3, AMACR, ZEB2, CHD3, |
| | | LRRC9, FLNC, HJURP, OPTN, CDO1, PARG, FAM153A, SCUBE2, |
| | | BTN3A1, BTN3A3, HMOX1, CSTA, SLFN11, ODC1, MEX3A, |
| | | PODXL2, EI24, KHDRBS3, VTCN1, CLDN4, LRG1, COL1A1, |
| | | DDX21, CYTH2, AP2S1, APELA, TLR1, OR51F1, SERTM1, WISP1, |
| | | CD37, S100A2, TSEN15, CSGALNACT1, LAMP5, VCAM1, GPR171, |
| | | ARHGEF1, POTEC, CAMK2N1, VWA2, MUC16, SYT6, CYP4F11, |
| | | IKZF1, PPP1R14B, IRF1, CD70, GPT2, PDAP1, ABCC11, CTTN, |
| | | APLN, KRT14, KLHL5, ITPR1, ZNF608, PRKCB, MCM7, RLIM, |
| | | RSPO3, MFAP5, CGN, COL8A1, BCHE, EIF3B, MKRN1, FABP4, |
| | | NPY4R, ANXA8, ZNF556, NRK, ZCCHC13, CLDN10, CDH19, |
| | | COLAA2, WDR93, AKAP2, FAM155A |

In some embodiments, the plurality of targets selected from the targets listed in Table 2 comprises or consists of at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 targets, or a range defined by any two of the preceding values (e.g., 2-215, 5-215, 10-215, 20-215, 50-215, 100-215, 10-20 or 5-10) selected from the 215 targets listed in Table 2.11.

TABLE 2.11

| Number | Accuracy | Targets |
|---|---|---|
| 215 | 100% | ABCC11, ACOXL, ACTC1, AKAP2, AMACR, AMD1, ANXA8, |
| | | AP2S1, APELA, APLN, AQP9, ARHGEF1, ATP1B2, B3GNT5, |
| | | BCHE, BTN3A1, BTN3A3, C12orf75, C1S, CACNA1D, CAMK2N1, |
| | | CAMK4, CBS, CCDC169, CD200, CD37, CD3D, CD70, CDH11, |
| | | CDH19, CDH7, CDO1, CENPF, CFB, CGN, CHD3, CHRM3, CHST9, |
| | | CKM, CLDN10, CLDN4, CLSTN2, COL1A1, COL3A1, COL4A2, |
| | | COL8A1, CP, CPA6, CPLX3, CSGALNACT1, CSTA, CTTN, |
| | | CYP4F11, CYP4F2, CYTH2, DDX21, DPT, EDN3, EI24, EIF3B, |
| | | FABP4, FAM111B, FAM153A, FAM155A, FAP, FAR2P1, FILIP1L, |
| | | FLNC, GABRD, GATA3, GBP1, GCNT1, GFRA3, GJB2, GOLGA8A, |
| | | GPR158, GPR171, GPT2, GTF2I, HDAC9, HIST1H1E, HIST1H2AD, |
| | | HJURP, HLA-DMB, HLA-F, HLF, HMOX1, HOXA-AS3, HS6ST2, |
| | | IKZF1, IKZF3, IRF1, ITGB6, ITGBL1, ITPR1, KHDRBS3, KL, |
| | | KLHL5, KLK12, KRT14, LAMP5, LGR5, LINC00668, LRG1, LRRC9, |
| | | LY6K, MAOB, MCM7, MEIS1, MEX3A, MFAP5, MKRN1, MORC2, |
| | | MUC1, MUC12, MUC15, MUC16, MUC2, MYL2, MYOC, MYOM1, |
| | | NPM1, NPY4R, NRK, ODC1, ONECUT2, OPTN, OR2L13, OR51F1, |
| | | OR52N5, ORM1, OS9, OXCT1, P2RY14, PABPC3, PARG, |
| | | PCDHA12, PDAP1, PDE11A, PICK1, PIGR, PLCXD3, PLEK, PMM2, |
| | | PNMA1, PODXL2, POTEB3, POTEC, POTED, PPP1R14B, PPP1R18, |
| | | PRKCB, PRKG2, PYHIN1, RAP1A, REXO2, RIMS1, RLIM, RND3, |
| | | RNPS1, RPAIN, RPL14, RPL22, RPS3, RRM2, RSPO3, S100A2, |
| | | S100A9, S100P, SCUBE2, SERPINB5, SERTM1, SET, SKA3, |
| | | SLC39A7, SLC44A5, SLC5A4, SLFN11, SMS, SNAP91, SPINK1, |
| | | SRPX2, SSRP1, STAC, STAU2-AS1, SUPT5H, SYCE1, SYT6, |
| | | TBC1D3B, TBX4, THBS4, TLR1, TNNI1, TPM2, TSEN15, TSPYL2, |
| | | TUBB4A, TUFM, UGDH, UGT2B4, UPK1A, UQCRH, USP17L10, |
| | | VCAM1, VSTM2L, VTCN1, VWA2, WDR93, WIF1, WISP1, |
| | | ZC3H13, ZCCHC13, ZEB2, ZNF556, ZNF608 |

In some embodiments of the methods disclosed above and elsewhere herein, one or more of the following is applicable. In some embodiments, the expression level of said target is reduced expression of said target. In some embodiments, the expression level of said target is increased expression of said target. In some embodiments, the level of expression of said target is detected or determined by using a method selected from the group consisting of in situ hybridization, a PCR-based method, an array-based method, an immunohistochemical method, an RNA assay method and an immunoassay method. In some embodiments, the method comprises using a reagent selected from the group consisting of a nucleic acid probe, one or more nucleic acid primers, and an antibody. In some embodiments, the nucleic acid probe is a reporter probe and/or a capture probe. In some embodiments, the prostate cancer subtype is selected from the group consisting of Luminal Differentiated (LD), Luminal Proliferating (LP), Basal Immune (BI), and Basal Neuroendocrine-like (BN). In some embodiments, the reagent is selected from the group consisting of a nucleic acid probe, one or more nucleic acid primers, and an antibody. In some embodiments, the nucleic acid probe is a reporter probe and/or a capture probe. In some embodiments, the treatment regimen is surgery, chemotherapy, radiation therapy, immunotherapy/biological therapy, hormonal therapy, and/or photodynamic therapy. In some embodiments, the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, or celecoxicib.

In some embodiments, the method comprises subtyping the subject as having LD prostate cancer. In some embodiments, the method further comprises predicting the subject having LD prostate cancer as benefiting from radiotherapy and/or radical prostatectomy, optionally benefiting more than a subject having LP, BI or BN prostate cancer. In some embodiments, the method further comprises prescribing and/or administering primary radiotherapy and/or radical prostatectomy. In some embodiments, the method further comprises predicting the subject having LD prostate cancer as not benefiting from docetaxel in addition to ADT. In some embodiments, the method further comprises not prescribing and/or not administering docetaxel in addition to ADT. In some embodiments, the method further comprises characterizing the LD prostate cancer as one or more of: androgen receptor (AR) driven; having high expression levels of prostate terminal differentiation markers, optionally higher than LP, BI or BN prostate cancer; having lower metastatic potential, optionally wherein the metastatic potential is lower than LP and BI prostate cancer; and/or being sensitive to ADT. In some embodiments, the method further comprises prescribing and/or administering ADT.

In some embodiments, the method comprises subtyping the subject as having LP prostate cancer. In some embodiments, the method further comprises predicting the subject having LP prostate cancer as benefiting from: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel. In some embodiments, the method further comprises prescribing and/or administering: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel. In some embodiments, the method further comprises predicting the subject having LP prostate cancer as benefiting from docetaxel in addition to ADT. In some embodiments, the method further comprises prescribing and/or administering docetaxel in addition to ADT. In some embodiments, the method further comprises characterizing the LP prostate cancer as one or more of: androgen receptor (AR) driven; having high expression levels of proliferation markers, optionally higher than LD, BI and BN prostate cancer; having higher metastatic potential, optionally higher than LD prostate cancer; insensitive to ADT; and/or sensitive to taxane-based chemotherapy and androgen receptor signaling inhibitors (ARSI). In some embodiments, the method further comprises not prescribing and/or not administering ADT. In some embodiments, the method further comprises prescribing and/or administering a taxane-based chemotherapy and/or an ARSI. In some embodiments, the method further comprises predicting the subject having LP prostate cancer as benefiting less from primary radiotherapy and/or radical prostatectomy than a subject with LD prostate cancer. In some embodiments, the method further comprises g predicting the subject having LP prostate cancer as benefiting more from primary radiotherapy than a subject with BN prostate cancer.

In some embodiments, the method comprises subtyping the subject as having LD prostate cancer, or, comprising subtyping the subject as having LP prostate cancer. In some embodiments, the method further comprises predicting the subject having LD or LP prostate cancer as not benefiting from higher dose primary radiotherapy, optionally wherein the higher dose primary radiotherapy is 79 Gy. In some embodiments, the method further comprises not prescribing and/or not administering a higher dose primary radiotherapy, optionally wherein the higher dose primary radiotherapy is 79 Gy. In some embodiments, the method further comprises prescribing and/or administering a lower dose primary radiotherapy, optionally wherein the lower dose primary radiotherapy is 70 Gy. In some embodiments, the method further comprises predicting the subject having LD or LP prostate cancer as not benefitting from long term adjuvant ADT, optionally wherein the long term ADT is 28 months. In some embodiments, the method further comprises not prescribing and/or not administering long term adjuvant ADT, optionally wherein the long term ADT is 28 months. In some embodiments, the method further comprises prescribing and/or administering short term adjuvant ADT, optionally wherein the short term ADT is 4 months. In some embodiments, the method further comprises predicting the subject having LD or LP prostate cancer as benefiting from the addition of long term ADT to salvage RT in combination with following biochemical recurrence, optionally wherein long term ADT is 24 months. In some embodiments, the method further comprises prescribing and/or administering long term ADT in addition to salvage RT following biochemical recurrence, optionally wherein long term ADT is 24 months. In some embodiments, the method further comprises predicting the subject having LD or LP prostate cancer as benefiting from abiraterone acetate. In some embodiments, the method further comprises prescribing and/or administering abiraterone acetate.

In some embodiments, the method comprises subtyping the subject as having BN prostate cancer. In some embodiments, the method further comprises predicting the subject having BN prostate cancer as benefiting from, an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib. In some embodiments, the method further comprises prescribing and/or administering: an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib. In some embodiments, the method further comprises predicting the subject having BN prostate cancer as not benefiting from docetaxel in addition to ADT. In some embodiments, the method further comprises not prescribing and/or not administering docetaxel in addition to ADT. In some embodiments, the method further comprises predicting the subject having BN prostate cancer as not benefiting from abiraterone acetate. In some embodiments, the method further comprises not prescribing and/or not administering abiraterone acetate. In some embodiments, the method further comprises predicting the subject having BN prostate cancer as benefiting less from primary radiotherapy and/or radical prostatectomy than a subject with LD prostate cancer. In some embodiments, the method further comprises predicting the subject having BN prostate cancer as benefiting less from primary radiotherapy than a subject with LD, BI or LP prostate cancer. In some embodiments, the method further comprises characterizing the BN prostate cancer as one or more of: non-AR driven; having the low expression of prostate terminal differentiation markers, optionally lower than LD, LP and BI prostate cancer; high expression of markers of a suppressed tumor immune microenvironment, optionally higher than LD, LP and BI prostate cancer; being resistant to ADT; and/or being sensitive to platinum and vinca alkaloid chemotherapies. In some embodiments, the method further comprises prescribing and/or administering a platinum and/or a vinca alkaloid chemotherapy. In some embodiments, the method further comprises not prescribing and/or not administering ADT.

In some embodiments, the method comprises subtyping the subject as having BI prostate cancer. In some embodiments, the method further comprises predicting the subject having BI prostate cancer as benefiting from: a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin. In some embodiments, the method further comprises prescribing and/or administering, a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin. In some embodiments, the method further comprises predicting the subject having BI prostate cancer as not benefiting from docetaxel in addition to ADT. In some embodiments, the method further comprises not prescribing and/or not administering docetaxel in addition to ADT. In some embodiments, the method further comprises predicting the subject having BI prostate cancer as benefiting less from primary radiotherapy and/or radical prostatectomy than a subject with LD prostate cancer. In some embodiments, the method further comprises predicting the subject having BI prostate cancer as benefiting more from primary radiotherapy than a subject with BN prostate cancer. In some embodiments, the method further comprises characterizing the BI prostate cancer as one or more of: non-AR driven; having elevated expression of other sex steroid transcription factors, optionally estrogen receptor, glucocorticoid receptor and progesterone receptors; sensitive to ADT; having high expression of markers of an activated tumor immune microenvironment, optionally higher than LD, LP and BN prostate cancer; having higher metastatic potential, optionally higher than LD prostate cancer; and/or sensitive to radiotherapy, protein kinase inhibitors and immune-checkpoint therapy. In some embodiments, the method further comprises prescribing and/or administering ADT, radiotherapy, a protein kinase inhibitor and/or immune-checkpoint therapy.

In some embodiments, the method comprises subtyping the subject as having BN prostate cancer, or, comprising subtyping the subject as having BI prostate cancer. In some embodiments, the method further comprises predicting the subject having BN or BI prostate cancer as benefiting from higher dose primary radiotherapy, optionally wherein the higher dose primary radiotherapy is 79 Gy. In some embodiments, the method further comprises prescribing and/or administering a higher dose primary radiotherapy, optionally wherein the higher dose primary radiotherapy is 79 Gy. In some embodiments, the method further comprises not prescribing and/or not administering a lower dose primary radiotherapy, optionally wherein the lower dose primary radiotherapy is 70 Gy. In some embodiments, the method further comprises predicting the subject having BN or BI prostate cancer as benefiting from long term adjuvant ADT, optionally wherein the long term ADT is 28 months. In some embodiments, the method further comprises prescribing and/or administering long term adjuvant ADT, optionally wherein the long term ADT is 28 months. In some embodiments, the method further comprises not prescribing and/or not administering short term adjuvant ADT, optionally wherein the short term ADT is 4 months. In some embodiments, the method further comprises predicting the subject having BN or BI prostate cancer as not benefiting from the addition of long term ADT to salvage RT in combination with following biochemical recurrence, optionally wherein long term ADT is 24 months. In some embodiments, the method further comprises not prescribing and/or administering long term ADT in addition to salvage RT following biochemical recurrence, optionally wherein long term ADT is 24 months.

In some embodiments, the sample or biological sample is a biopsy, urine sample, a blood sample or a prostate tumor sample. In some embodiments, the blood sample is plasma, serum, or whole blood. In some embodiments, the subject is a human. In some embodiments, the level of expression is increased or reduced compared to a control. In some embodiments, the measuring the level of expression comprises measuring the level of an RNA transcript. In some embodiments, the plurality of targets are nucleic acid targets. In some embodiments, the plurality of nucleic acid targets comprises a coding target. In some embodiments, the coding target is an exonic sequence. In some embodiments, the plurality of nucleic acid targets comprises a non-coding target. In some embodiments, the non-coding target comprises an intronic sequence or partially overlaps an intronic sequence. In some embodiments, the non-coding target comprises an intergenic sequence. In some embodiments, the non-coding target comprises a sequence within the untranslated region (UTR) or partially overlaps with a UTR sequence. In some embodiments, the plurality of nucleic acid targets comprise a DNA sequence. In some embodiments, the plurality of nucleic acid targets comprise an RNA sequence. In some embodiments, the embodiment further comprises sequencing the plurality of nucleic acid targets. In some embodiments, the embodiment further comprises hybridizing the plurality of nucleic acid targets to a solid support. In some embodiments, the solid support is a bead or array.

Before the present disclosure is described in further detail, it is to be understood that this disclosure is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present disclosure.

### Targets

The methods disclosed herein often comprise performing or having performed an assay of the expression level of a plurality of targets (also referred to as “target genes” or “gene targets”). The plurality of targets may comprise coding targets and/or non-coding targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets may be differentially expressed. In some instances, a plurality of probe selection regions (PSRs) is differentially expressed.

In some instances, the plurality of targets comprises one or more gene targets selected from Table 2. In some instances, the plurality of targets comprises at least about 2, at least about 3, at least about 4, at least about 5, at least about 6, at least about 7, at least about 8, at least about 9, or at least about 10 targets selected from Table 2. In some embodiments, the plurality of targets does not include one or more of the targets listed in Table 2. In some embodiments, the plurality of targets includes not more than 200, 175, 150, 125, 100, 75, 50 or 25 of the targets listed in Table 2. In some embodiments, the plurality of targets comprises or consists of at least 2, 3, 4, 5, 6, 7, 8, 9, 10, 11, 12, 13, 14, 15, 20, 25, 30, 40, 50, 75, 100, 200, 210 or 215 targets, or a range defined by any two of the preceding values, (e.g., 2-20, 5-20, 10-20, 2-30, 5-30, 10-30, 20-30, 2-40, 5-40, 10-40, 20-40, 30-40, 2-50, 5-50, 10-50, 20-50, 40-50, 2-60, 5-60, 10-60, 20-60, 50-60, 2-70, 5-70, 10-70, 20-70, 50-70, 2-80, 5-80, 10-80, 20-80, 50-80, 2-90, 5-90, 10-90, 20-90, 50-90, 2-100, 5-100, 10-100, 20-100, 50-100, 2-110, 5-110, 10-110, 20-110, 50-110, 100-110, 2-215, 5-215, 10-215, 20-215, 50-215, 100-215, 10-20 or 5-10) selected from the targets listed in Table 2, 2.1, 2.2, 2.3, 2.4, 2.5, 2.6, 2.7, 2.8, 2.9, 2.10, or 2.11. Tables 2.1-2.10 list subsets of targets of those listed in Table 2 and 2.11 (both tables list the same targets) which can be used in the embodiments disclosed herein. Tables 2.1-2.10 list the accuracy of classifying samples as LD, LP, BI or BN as compared to using all 215 targets listed in Tables 2 and 2.11. That is to say that if the 20 targets listed in Table 2.1 are used, 85% of the samples will be assigned the same subtype had all 215 targets listed in Tables 2 and 2.11 been used. If the additional 10 targets listed in Table 2.2 are added to those listed in Table 2.1 (a total of 30 targets), 88% of the samples will be assigned the same subtype had all 215 targets listed in Tables 2 and 2.11 been used. As expected, the more targets used, the greater the consistency between the subset of targets listed in Tables 2.1-2.10 and the results obtained using all 215 targets listed in Table 2. Table 2.11 lists all the targets listed in Table 2, and thus there is 100% agreement between using those targets in Table 2.11 and Table 2. In some embodiments, the plurality of targets includes not more than 215, 210, 200, 175, 150, 125, 110, 100, 90, 80, 70, 60, 50, 40, 30, 20, 15, 14, 13, 12, 11, 10, 9, 8, 7, 6, or 5 of the targets listed in Table 2, 2.1, 2.2, 2.3, 2.4, 2.5, 2.6, 2.7, 2.8, 2.9, 2.10, or 2.11. In some embodiments, the plurality of targets have an absolute value of the coefficient value in Table 2 of at least 0.10000, 0.12500, 0.15000, 0.17500, 0.20000, 0.22500, 0.25000, 0.27500, 0.30000, 0.32500, 0.35000, 0.37500, 0.40000, 0.42500, 0.45000, 0.47500, 0.50000, 0.52500, 0.55000, 0.57500, 0.60000, 0.62500, 0.65000, 0.67500, 0.70000, 0.72500, 0.75000, 0.77500, 0.80000, 0.82500, 0.85000, 0.87500, 0.90000, 0.92500, 0.95000, 0.97500, 1.00000, 1.50000, 2.00000, 2.50000, 3.00000, 3.50000, or 4.00000. For example, in some embodiments, the plurality of targets comprises or consists of those targets with coefficient value of 0.25000 or greater, or −0.2500 or less (i.e. a coefficient value with an absolute value of at least 0.25000).

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15 or more targets selected from a Random Forest (RF) classifier. The RF classifier may be an RF2, and RF3, or an RF4 classifier. The RF classifier may be an RF15 classifier (e.g., a Random Forest classifier with 15 targets).

A RF classifier of the present disclosure may comprise two or more targets comprising two or more targets selected from Table 2.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier. The SVM classifier may be an SVM2 classifier.

A SVM classifier of the present disclosure may comprise two or more targets comprising two or more targets selected from Table 2.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100, 200 or more targets selected from a KNN classifier.

The KNN classifier may be a KNN2 classifier. A KNN classifier of the present disclosure may comprise two or more targets comprising two or more targets selected from Table 2.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100, 200 or more targets selected from a NB classifier.

The NB classifier may be a NB2 classifier. An NB classifier of the present disclosure may comprise two or more targets comprising two or more targets selected from Table 2.

The plurality of targets may comprise one or more targets selected from a recursive Partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100, 200 or more targets selected from an Rpart classifier.

The Rpart classifier may be an Rpart2 classifier. An Rpart classifier of the present disclosure may comprise two or more targets comprising two or more targets selected from Table 2.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15 or more targets selected from a high dimensional discriminate analysis (HDDA) classifier.

In some embodiments, the sample or biological sample is a biopsy, urine sample, a blood sample or a prostate tumor sample. In some embodiments, the blood sample is plasma, serum, or whole blood. In some embodiments, the subject is a human. In some embodiments, the level of expression is increased or reduced compared to a control. In some embodiments, the measuring the level of expression comprises measuring the level of an RNA transcript. In some embodiments, the plurality of targets are nucleic acid targets. In some embodiments, the plurality of nucleic acid targets comprises a coding target. In some embodiments, the coding target is an exonic sequence. In some embodiments, the plurality of nucleic acid targets comprises a non-coding target. In some embodiments, the non-coding target comprises an intronic sequence or partially overlaps an intronic sequence. In some embodiments, the non-coding target comprises an intergenic sequence. In some embodiments, the non-coding target comprises a sequence within the untranslated region (UTR) or partially overlaps with a UTR sequence. In some embodiments, the plurality of nucleic acid targets comprise a DNA sequence. In some embodiments, the plurality of nucleic acid targets comprise an RNA sequence. In some embodiments, the embodiment further comprises sequencing the plurality of nucleic acid targets. In some embodiments, the embodiment further comprises hybridizing the plurality of nucleic acid targets to a solid support. In some embodiments, the solid support is a bead or array.

### Probes/Primers

The present disclosure provides for a probe set for diagnosing, prognosing, treating, monitoring and/or predicting a status or outcome of a prostate cancer in a subject comprising a plurality of probes, wherein (i) the probes in the set are capable of detecting an expression level of at least one target selected from Table 2; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present disclosure may range in length from about 15 nucleotides to the full length of the coding target or non-coding target. In one embodiment of the disclosure, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. In certain embodiments, the probe set comprises a combination of a coding target and non-coding target.

In some embodiments, disclosed is a probe set for use in the method of any embodiment disclosed herein. In some embodiments, the probe set comprises or consists of a plurality of probes, where the probes in the set are used for detecting the expression level of a plurality of nucleic acid targets in a sample from the subject. In some embodiments, disclosed is a probe set for subtyping, prognosing and/or predicting benefit from prostate cancer therapy of a prostate cancer in a subject. In some embodiments, the probe set comprises or consists of a plurality of probes, where the probes in the set are used for detecting the expression level of a plurality of nucleic acid targets in a sample from the subject. In some embodiments, the probes of the probe set hybridize to the nucleic acid targets. In some embodiments, the plurality of nucleic acid targets comprise or consist of a plurality of targets selected the targets in Table 2. In some embodiments, the plurality of targets comprises or consists of at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 targets selected from the targets listed in Table 2, 2.1, 2.2, 2.3, 2.4, 2.5, 2.6, 2.7, 2.8, 2.9, 2.10, or 2.11. In some embodiments, the plurality of targets includes not more than 215, 210, 200, 175, 150, 125, 110, 100, 90, 80, 70, 60, 50, 40, 30, 20, 15, 14, 13, 12, 11, 10, 9, 8, 7, 6, or 5 of the targets listed in Table 2, 2.1, 2.2, 2.3, 2.4, 2.5, 2.6, 2.7, 2.8, 2.9, 2.10, or 2.11. In some embodiments of the probe set, the plurality of nucleic acid targets comprises a coding target. In some embodiments of the probe set, the coding target is an exonic sequence. In some embodiments of the probe set, the plurality of nucleic acid targets comprises a non-coding target. In some embodiments of the probe set, the non-coding target comprises an intronic sequence or partially overlaps an intronic sequence. In some embodiments of the probe set, the non-coding target comprises an intergenic sequence. In some embodiments of the probe set, the non-coding target comprises a sequence within the untranslated region (UTR) or partially overlaps with a UTR sequence. In some embodiments of the probe set, the plurality of nucleic acid targets comprise a DNA sequence. In some embodiments of the probe set, the plurality of nucleic acid targets comprise an RNA sequence.

In some embodiments, the probe set comprise a plurality of target sequences that hybridize to at least about 5 targets selected from Table 2. Alternatively, the probe set comprise a plurality of target sequences that hybridize to at least about 10 targets from Table 2. In some embodiments, the probe set comprise a plurality of target sequences that hybridize to at least about 15 targets selected from Table 2. In some embodiments, the probe set comprise a plurality of target sequences that hybridize to at least about 20 targets selected from Table 2. In some embodiments, the probe set comprise a plurality of target sequences that hybridize to at least about 30 targets selected from Table 2. In some embodiments, the probe set comprise a plurality of target sequences that hybridize to at least about 100 targets selected from Table 2. In some embodiments, the probe set comprise a plurality of target sequences that hybridize to all 215 targets in Table 2.

In some embodiments, the probe set comprises or consists of a plurality of target sequences that hybridize to at least about 5, 10, 15, 20, 25, 30, 40, 50, 60, 70, 80, 90, 100, 110, 150, 200, 210, or 215 targets selected from Tables 2.1-2.11.

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the disclosure, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR

In one embodiment, the primers or primer pairs, when used in an amplification reaction, specifically amplify at least a portion of a nucleic acid sequence of a target selected from Table 2 (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the disclosure comprise at least 20 consecutive bases of the nucleic acid sequence of a target selected from Table 2 or a complement thereto. The polynucleotides may comprise at least 21, 22, 23, 24, 25, 27, 30, 32, 35 or more consecutive bases of the nucleic acids sequence of a target selected from Table 2, as applicable.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

In other embodiments, the level of expression of the plurality of targets is determined by using a Nanostring nCounter. A Nanostring nCounter can be used for multiplexed measurement of nucleic acids with high levels of precision and sensitivity using fluorescent color-coded barcode probes. The probes hybridize to approximately 100 bases and can be multiplexed to detect up to 800 different targets in a single reaction. The nCounter technology uses molecular “barcodes” and single molecule imaging to detect and count hundreds of unique transcripts in a single reaction at high sensitivity (<1 copy per cell).

In some embodiments, disclosed herein is a system for analyzing a cancer, comprising: (a) a probe set disclosed in embodiments herein; and (b) a computer model or algorithm for analyzing an expression level and/or expression profile of the target hybridized to the probe in a sample from a subject suffering from prostate cancer. In some embodiments, the system further comprises a computer model or algorithm for correlating the expression level or expression profile with disease state or outcome. In some embodiments, the system further comprises a computer model or algorithm for designating a treatment modality for the subject. In some embodiments, the system further comprises a computer model or algorithm for normalizing expression level or expression profile of the target sequences.

### Diagnostic Samples

Diagnostic samples for use with the systems and in the methods of the present disclosure comprise nucleic acids suitable for providing RNAs expression information. In principle, the biological sample from which the expressed RNA is obtained and analyzed for target sequence expression can be any material suspected of comprising prostate cancer tissue or cells. The diagnostic sample can be a biological sample used directly in a method of the disclosure. Alternatively, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

In some embodiments, the sample or biological sample is a biopsy, urine sample, a blood sample or a prostate tumor sample. In some embodiments, the blood sample is plasma, serum, or whole blood. In some embodiments, the subject is a human. In some embodiments, the level of expression is increased or reduced compared to a control. In some embodiments, the measuring the level of expression comprises measuring the level of an RNA transcript. In some embodiments, the plurality of targets are nucleic acid targets. In some embodiments, the plurality of nucleic acid targets comprises a coding target. In some embodiments, the coding target is an exonic sequence. In some embodiments, the plurality of nucleic acid targets comprises a non-coding target. In some embodiments, the non-coding target comprises an intronic sequence or partially overlaps an intronic sequence. In some embodiments, the non-coding target comprises an intergenic sequence. In some embodiments, the non-coding target comprises a sequence within the untranslated region (UTR) or partially overlaps with a UTR sequence. In some embodiments, the plurality of nucleic acid targets comprise a DNA sequence. In some embodiments, the plurality of nucleic acid targets comprise an RNA sequence. In some embodiments, the embodiment further comprises sequencing the plurality of nucleic acid targets. In some embodiments, the embodiment further comprises hybridizing the plurality of nucleic acid targets to a solid support. In some embodiments, the solid support is a bead or array.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly MA, High Pure FFPE RNA Micro Kit, Roche Applied Science, Indianapolis, IN). RNA can be extracted from frozen tissue sections using TRIzol (Invitrogen, Carlsbad, CA) and purified using RNeasy Protect kit (Qiagen, Valencia, CA). RNA can be further purified using DNAse I treatment (Ambion, Austin, TX) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, CA).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 200 or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 2, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 2, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® 1I, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, CA), including the WT-Ovation™System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, CA), Genisphere, Inc. (Hatfield, PA), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, CA), SmartCycler® 9600 or GeneXpert® Systems (Cepheid, Sunnyvale, CA), ABI 7900 HT Fast Real Time PCR system (Applied Biosystems, Foster City, CA), LightCycler® 480 System (Roche Molecular Systems, Pleasanton, CA), xMAP 100 System (Luminex, Austin, TX) Solexa Genome Analysis System (Illumina, Hayward, CA), OpenArray Real Time qPCR (BioTrove, Woburn, MA) and BeadXpress System (Illumina, Hayward, CA).

Detection and/or Quantification of Target Sequences

Any method of detecting and/or quantitating the expression of the encoded target sequences can in principle be used in the disclosure. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, fluorescent barcodes and single molecule imaging (e.g., Nanostring nCounter), bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for ORT-PCR Analysis

Reverse transcription can be performed by any method known in the art. For example, reverse transcription may be performed using the Omniscript kit (Qiagen, Valencia, CA), Superscript III kit (Invitrogen, Carlsbad, CA), for RT-PCR. Target-specific priming can be performed in order to increase the sensitivity of detection of target sequences and generate target-specific cDNA.

### TaqMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 2 or a product derived thereof can be used. Desirably, an array may be specific for 5, 10, 15, 20, 25, 30 or more of transcripts of a target selected from Table 2. Expression of these sequences may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, CA.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present disclosure, the Cancer Prognostic array is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Performing or having performed an assay of the expression level for a plurality of targets may comprise the use of an algorithm or classifier. Array data can be managed, classified, and analyzed using techniques known in the art. Performing or having performed an assay of the expression level for a plurality of targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

Alternatively, performing or having performed an assay of the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naïve Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naïve Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Cancer

The systems, compositions and methods disclosed herein may be used to diagnosis, monitor and/or predict the status or outcome of a cancer. Generally, a cancer is characterized by the uncontrolled growth of abnormal cells anywhere in a body. The abnormal cells may be termed cancer cells, malignant cells, or tumor cells. Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1M0 or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

Alternatively, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage 1 often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than ⅓ of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

### Therapeutic Regimens

Diagnosing, prognosing, or monitoring a status or outcome of a cancer may comprise treating a cancer or preventing a cancer progression. In addition, diagnosing, prognosing, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camprothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some embodiments, the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, or celecoxicib.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (C02) lasers, argon lasers, and neodymium:yttrium-aluminum-garnet (Nd:YAG) lasers. Each of these can shrink or destroy tumors and can be used with endoscopes. C02 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For patients with high test scores consistent with systemic disease outcome after prostatectomy, additional treatment modalities such as adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride) can be designated. Such patients would likely be treated immediately with anti-androgen therapy alone or in combination with radiation therapy in order to eliminate presumed micro-metastatic disease, which cannot be detected clinically but can be revealed by the target sequence expression signature.

Such patients can also be more closely monitored for signs of disease progression. For patients with intermediate test scores consistent with biochemical recurrence only (BCR-only or elevated PSA that does not rapidly become manifested as systemic disease only localized adjuvant therapy (e.g., radiation therapy of the prostate bed) or short course of anti-androgen therapy would likely be administered. For patients with low scores or scores consistent with no evidence of disease (NED) adjuvant therapy would not likely be recommended by their physicians in order to avoid treatment-related side effects such as metabolic syndrome (e.g., hypertension, diabetes and/or weight gain), osteoporosis, proctitis, incontinence or impotence. Patients with samples consistent with NED could be designated for watchful waiting, or for no treatment. Patients with test scores that do not correlate with systemic disease but who have successive PSA increases could be designated for watchful waiting, increased monitoring, or lower dose or shorter duration anti-androgen therapy.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty or more of the target sequences corresponding to a target selected from Table 2, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### Subtyping

The inventors of the present disclosure discovered multiple subtypes of prostate cancer, including, for example, Luminal Differentiated (LD), Luminal Proliferating (LP), Basal Immune (BI), and Basal Neuroendocrine-like (BN). Molecular subtyping is a method of classifying prostate cancers into one of multiple genetically-distinct categories, or subtypes. Each subtype responds differently to different kinds of treatments, and some subtypes indicate a higher risk of recurrence. As described herein, each subtype has a unique molecular and clinical fingerprint.

Differential expression analysis one or more of the targets listed in Table 2 allow for the identification of the molecular subtype of a prostate cancer.

In some instances, the molecular subtyping methods of the present disclosure are used in combination with other biomarkers, like tumor grade and hormone levels, for analyzing the prostate cancer.

### Clinical Associations and Patient Outcomes

Molecular subtypes of the present disclosure have distinct clinical associations. Clinical associations that correlate to molecular subtypes include, for example, preoperative serum PSA, Gleason score (GS), extraprostatic extension (EPE), surgical margin status (SM), lymph node involvement (LNI), and seminal vesicle invasion (SVI).

In some embodiments, molecular subtypes of the present disclosure are used to predict patient outcomes such as biochemical recurrence (BCR), metastasis (MET) and prostate cancer death (PCSM) after radical prostatectomy. For example, Luminal Differentiated prostate cancer patients consistently have significantly better outcomes for all endpoints compared to Luminal Proliferating, Basal Immune and Basal NE-like subtypes (see Example 1).

### Treatment Response Prediction

In some embodiment, the molecular subtypes of the present disclosure are useful for predicting prostate cancer response to hormone therapy (e.g., androgen deprivation therapy). In other embodiments, the molecular subtypes of the present disclosure are useful for predicting prostate cancer response to radiation therapy (RT). In yet other embodiments, the molecular subtypes of the present disclosure are useful for predicting prostate cancer response to the addition of ADT to salvage radiotherapy. In still other embodiments, the molecular subtypes of the present disclosure are useful for predicting prostate cancer response to chemotherapy.

### EXAMPLES

### Example 1: Development and Validation of a Prostate Subtyping Classifier for Prostate Cancer and Prediction of Treatment Response

A genomic-based prostate subtyping classifier (PSC) for prostate cancer was developed as follows. De-identified transcriptome profiles collected from clinical use of the Decipher prostate genomic classifier (Decipher Biosciences, San Diego, CA) in 12,000 men at initial diagnosis and 20,000 men treated with radical prostatectomy were used to train a novel Prostate Subtyping Classifier (PSC) model. An additional 32,416 transcriptomes from clinical use of the Decipher prostate genomic classifier not used for model training were used for model evaluation. From these patients, four clusters of prostate cancers were identified with distinct biological characteristics based on expression patterns of a curated panel of signatures and individual genes (n=21). A multinomial logistic regression model was trained to reproduce the clusters as a single sample classifier. The four clusters (i.e., PSC subtypes) were named as Luminal Differentiated (LD), Luminal Proliferating (LP), Basal Immune (BI) and Basal Neuroendocrine-like (BN). Data from large, multi-institutional retrospective cohorts (n=855) and several randomized Phase III clinical trials (n=992), which had long-term follow-up, treatment and outcomes data were used to validate the model and test its associations with outcomes and response to several therapies, including, hormone therapy, radiation therapy and chemotherapy.

The Decipher prostate genomic classifier assay utilizes the Human Exon 1.0 ST oligonucleotide microarray (ThermoFisher, Santa Clara, CA) to measure the expression of 46,050 genes and non-coding RNA transcripts. Microarray processing was performed in a CLIA-certified clinical operations laboratory (Decipher Biosciences, Inc, San Diego, CA). Microarrays were normalized using Single Channel Array Normalization. (Piccolo et al. Genomics. 2012; 100(6):337-344.)

### PSC Model Development

Expression data was filtered to select for 18,696 well-annotated genes. These represent genes whose exons are present in all known transcript isoforms expressed from a single gene locus. Next, a signal to noise filter was applied to remove genes with a standard error smaller than 0.1 in the training cohort or had an intraclass-correlation coefficient (ICC, a measure of intra-sample reproducibility)<0.5 as measured in previously conducted analytical validation experiments for the Decipher assay. This resulted in a total of 6,925 genes available for single sample classifier training. To ensure model fidelity in both biopsy and RP tissue specimens as well as for both prospectively collected vs retrospective archived samples, genes that had a distributional difference larger than 0.2 among the three cohorts were further filtered out. The distributional difference was defined as the maximum absolute difference between the cumulative distribution function (i.e., Kolmogorov-Smirnov statistic). The distribution difference was calculated between the 12,000-biopsy vs 20,000 RP samples (i.e., the 32,000-training cohort) vs a large retrospective RP cohort (n=1230, Erho et al. 2013, PloS one; Klein, et al. 2014, European Urology).

To train the model, an initial clustering was performed with the patient samples based on a panel of 21 seed features (Table 1), which represent pathways believed to be important sources of biological variance in prostate cancer. Hierarchical clustering (Ward linkage method with Pearson correlation distance metric) was performed on the 32,000 samples based on the percentile of 21 features. Four major clusters of patients were observed which formed the basis for a second round of modeling. Samples in the training cohort were then labeled based on the combination of the two rounds of clustering. Using these initial sample labels we further selected 60 genes over-expressed in pairwise comparisons (Wilcoxon test) for each of the four sample clusters (as compared to the remainders). This resulted in the identification of 215 unique genes, which were then used to fit a GLMNET multinomial logistic regression model, whose hyper parameters were selected via cross validation. All genes have non-zero coefficients in the final model (Table 2). For each single sample, the classifier outputs a probability of belonging to one of four classes and the class with the highest probability is reported as the sample prostate cancer subtype.

TABLE 1

| Cluster Seeding Gene/ | Cluster Seeding | |
|---|---|---|
| Signature Name | Biological Module | Reference |
| AR gene | Androgen receptor signaling | |
| AR Activity | Androgen receptor signaling | Spratt et al. Clin Cancer Res. 2019 Nov. |
| | | 15; 25(22): 6721-6730 |
| FOXA1 gene | Androgen receptor signaling | |
| KLK2 gene | Androgen receptor signaling | |
| KLK4 gene | Androgen receptor signaling | |
| AR Activity | Androgen receptor signaling | Kumar et al. Nat Med. 2016 April; 22(4): 369-78. |
| Stem-cell & basal cell | Basal/Neuroendocrine | Zhang et al. Nat Commun. 2016 Feb. |
| lineage signature | | 29; 7: 10798. |
| Immune 190 | Immune Activation | Korpal et al. Nat Commun. 2017 Jul. 24; 8(1): 103. |
| Cytotoxic T cells | Immune Activation | Jiang et al. Nat Med 24, 1550-1558 (2018) |
| Interferon gamma | Immune Activation | Jiang et al. Nat Med 24, 1550-1558 (2018) |
| ESTIMATE Immune | Immune Activation | Yoshihara et al. Nat Commun. 2013; 4: 2612 |
| Th2 T helper cell | Immune Activation | Şenbabaoǧlu et al. Genome Biol 17, 231 |
| signature | | (2016). |
| T regulatory signature | Immune Suppression | Charoentong et al. Cell Rep. 2017 Jan. |
| | | 3; 18(1): 248-262. |
| Myeloid-derived | Immune Suppression | Charoentong et al. Cell Rep. 2017 Jan. |
| suppressor cell | | 3; 18(1): 248-262. |
| Cell Cycle Progression | Proliferation/risk models | Cuzick et al. Lancet Oncol. 2011 |
| | | March; 12(3): 245-55. |
| Decipher | Proliferation/risk models | Erho et al. PLoS One. 2013 Jun. |
| | | 24; 8(6): e66855. |
| MKI67 gene | Proliferation/risk models | |
| TOP2A gene | Proliferation/risk models | |
| FOXM1 gene | Proliferation/risk models | |
| PTTG1 gene | Proliferation/risk models | |
| Risk model | Proliferation/risk models | Chin-Lee et al. PNAS. 2013 Apr. 9; 110(15): |
| | | 6121-6126. |

TABLE 2

| | Luminal | Luminal | Basal | Basal |
|---|---|---|---|---|
| | Differentiated | Proliferating | Immune | Neuroendocrine- |
| | (LD) | (LP) | (BI) | like (BN) |
| (Intercept) | −0.294756275 | −1.457563347 | −1.609586995 | 3.361906616 |
| ABCC11 | −0.3111334 | 0.020482542 | 0.214000711 | 0.076650147 |
| ACOXL | 1.208206918 | 0.24228379 | −0.974622809 | −0.475867899 |
| ACTC1 | 0.083073285 | 0.175895025 | −0.016327905 | −0.242640404 |
| AKAP2 | 0.03372906 | −0.066497114 | 0.070730156 | −0.037962102 |
| AMACR | 0.121318414 | 0.062197433 | −0.062212312 | −0.121303535 |
| AMD1 | 0.115019578 | 0.085297284 | −0.091025132 | −0.109291731 |
| ANXA8 | −0.442266522 | 0.058324145 | 0.081408618 | 0.302533759 |
| AP2S1 | 0.457272593 | 0.401589595 | −0.346384965 | −0.512477223 |
| APELA | 0.008610491 | 0.306268741 | −0.184472093 | −0.130407139 |
| APLN | −0.306227172 | 0.225450123 | 0.200346736 | −0.119569687 |
| AQP9 | −0.122512742 | 0.052846765 | −0.041122917 | 0.110788893 |
| ARHGEF1 | 0.998922901 | 0.132001944 | −0.474124249 | −0.656800595 |
| ATP1B2 | 0.172711674 | 0.307806022 | −0.111048036 | −0.36946966 |
| B3GNT5 | −0.449286265 | −0.124958298 | 0.310882094 | 0.26336247 |
| BCHE | −0.017981212 | −0.134674328 | −0.15675748 | 0.30941302 |
| BTN3A1 | 0.534686568 | 0.022877074 | −0.004696844 | −0.552866798 |
| BTN3A3 | −0.519007896 | −0.547315974 | 0.860806497 | 0.205517373 |
| C12orf75 | −0.083505222 | −0.103522354 | 0.031597576 | 0.15543 |
| C1S | 0.009522973 | −0.492993659 | 0.559694452 | −0.076223765 |
| CACNA1D | −0.015329282 | 0.164299604 | −0.078571594 | −0.070398729 |
| CAMK2N1 | 0.13441178 | 0.036842967 | 0.071372059 | −0.242626807 |
| CAMK4 | 0.280720254 | −0.255480551 | 0.002849132 | −0.028088835 |
| CBS | 0.199129956 | 0.370571767 | −0.299866809 | −0.269834914 |
| CCDC169 | 0.027403831 | 0.117257217 | −0.056403213 | −0.088257835 |
| CD200 | −0.141312562 | −0.15331432 | 0.159615294 | 0.135011589 |
| CD37 | −0.162375464 | −0.694050976 | 1.008249599 | −0.151823159 |
| CD3D | −0.413153952 | −0.664562402 | 1.453516825 | −0.375800472 |
| CD70 | 0.479393655 | −0.269412898 | −0.063720752 | −0.146260005 |
| CDH11 | −0.223669298 | −0.296037785 | 0.557063549 | −0.037356465 |
| CDH19 | 0.178692914 | −0.173132379 | 0.038418579 | −0.043979114 |
| CDH7 | −0.24183945 | −0.017951103 | 0.138256424 | 0.121534128 |
| CDO1 | 0.302916475 | −0.168987425 | −0.065707693 | −0.068221356 |
| CENPF | −4.850537721 | 1.712953679 | 1.670702324 | 1.466881718 |
| CFB | −0.012212252 | 0.054053075 | 0.116380338 | −0.158221162 |
| CGN | −0.183906833 | 0.346845347 | −0.252692698 | 0.089754184 |
| CHD3 | 0.268861902 | 0.370720227 | −0.3894969 | −0.250085229 |
| CHRM3 | 0.060868121 | 0.028233421 | −0.125588399 | 0.036486857 |
| CHST9 | −0.188202055 | −0.090347332 | 0.166011992 | 0.112537395 |
| CKM | 0.008088 | 0.067953906 | 0.012646003 | −0.088687909 |
| CLDN10 | 0.12048135 | −0.130770944 | −0.187481582 | 0.197771175 |
| CLDN4 | 0.229707706 | 0.348462025 | −0.124457634 | −0.453712096 |
| CLSTN2 | 0.100102721 | −0.092033843 | −0.047175377 | 0.039106499 |
| COL1A1 | −0.124060855 | 0.05908623 | 0.095837651 | −0.030863027 |
| COL3A1 | −0.150310929 | −0.137513454 | 0.219540694 | 0.068283689 |
| COL4A2 | −0.202732921 | −0.030210644 | 0.586918175 | −0.35397461 |
| COL8A1 | −0.301373327 | −0.104961345 | 0.207781783 | 0.198552888 |
| CP | −0.240589697 | 0.215968244 | 0.171905955 | −0.147284501 |
| CPA6 | 0.027779706 | −0.064042984 | −0.299539773 | 0.335803051 |
| CPLX3 | 0.094212757 | 0.076965436 | −0.043015866 | −0.128162326 |
| CSGALNACT1 | −0.007788517 | 0.236987393 | −0.136777525 | −0.092421352 |
| CSTA | −0.341238894 | −0.31597544 | 0.46050667 | 0.196707664 |
| CTTN | −0.285859781 | 0.446683823 | −0.219423186 | 0.058599144 |
| CYP4F11 | 0.331452201 | 0.247830019 | −0.300265031 | −0.279017189 |
| CYP4F2 | 0.198322201 | −0.050886717 | −0.107913061 | −0.039522423 |
| CYTH2 | −0.244162942 | 0.608409733 | −0.330536629 | −0.033710163 |
| DDX21 | −0.327828406 | 0.054406783 | 0.313458984 | −0.040037362 |
| DPT | 0.101064441 | −0.074451554 | −0.032541279 | 0.005928392 |
| EDN3 | 0.378578336 | 0.179038744 | −0.122941502 | −0.076598091 |
| EI24 | 0.413943602 | 0.679927795 | −0.937864203 | −0.156007194 |
| EIF3B | −0.389758201 | −0.12332781 | −0.021762208 | 0.534848218 |
| FABP4 | 0.187150963 | −0.250780927 | −0.043016387 | 0.106646351 |
| FAM111B | −0.895678244 | 0.670598275 | −0.024552971 | 0.24963294 |
| FAM153A | 0.778199885 | −0.126527984 | −0.09566623 | −0.556005671 |
| FAM155A | −0.347724829 | −0.140229328 | 0.175424193 | 0.312529965 |
| FAP | 0.063784882 | −0.352000572 | 0.217034772 | 0.071180918 |
| FAR2P1 | −0.022331286 | 0.076489383 | 0.029799705 | −0.083957801 |
| FILIP1L | −0.401196269 | −0.396947795 | 0.566153387 | 0.231990677 |
| FLNC | 0.249049406 | −0.120741231 | −0.115434542 | −0.012873634 |
| GABRD | −0.693558162 | −0.087238621 | 0.620611776 | 0.160185007 |
| GATA3 | −0.039704228 | −0.292849922 | 0.247311877 | 0.085242273 |
| GBP1 | −0.214036324 | 0.592102224 | 1.164403071 | −0.358264523 |
| GCNT1 | −0.02593809 | 0.176086172 | −0.142151356 | −0.007996727 |
| GFRA3 | 0.080345549 | −0.026089148 | −0.061310564 | 0.007054163 |
| GJB2 | 0.015313663 | −0.022177946 | −0.016119881 | 0.022984164 |
| GOLGA8A | 0.022020014 | 0.104926797 | −0.018968441 | −0.10797837 |
| GPR158 | −0.151727586 | −0.016203219 | 0.03977442 | 0.128156385 |
| GPR171 | 0.03744631 | −0.287162213 | 0.467261376 | −0.217545474 |
| GPT2 | 0.398568437 | −0.179891257 | −0.222286694 | 0.003609514 |
| GTF2I | −0.064763191 | 0.351758842 | −0.202209617 | −0.084786034 |
| HDAC9 | −0.195470712 | −0.181616828 | 0.225263106 | 0.151824434 |
| HIST1H1E | 0.007481143 | 0.037517838 | 0.011054368 | −0.056053349 |
| HIST1H2AD | −0.001954273 | 0.04670584 | 0.034375495 | −0.079127062 |
| HJURP | −2.049191129 | 0.672237977 | 0.659310156 | 0.717642996 |
| HLA-DMB | −0.018168645 | 0.17063145 | 0.131394365 | −0.28385717 |
| HLA-F | 0.761553044 | −0.860782488 | 1.055909657 | −0.956680214 |
| HLF | 0.109509904 | 0.013448687 | 0.067410755 | −0.190369347 |
| HMOX1 | −0.329173272 | −0.363488993 | 0.829910274 | −0.137248009 |
| HOXA-AS3 | 0.834079283 | −0.000158548 | −0.788665758 | −0.045254977 |
| HS6ST2 | −0.316615541 | −0.54301748 | 0.422803007 | 0.436830014 |
| IKZF1 | 0.012719387 | −0.466491898 | 0.82694466 | −0.373172149 |
| IKZF3 | −0.908308739 | −0.797774038 | 1.646436053 | 0.059646724 |
| IRF1 | 0.034226646 | −0.509386123 | 0.695701656 | −0.220542179 |
| ITGB6 | 0.531453846 | −0.41749979 | −0.203790987 | 0.089836931 |
| ITGBL1 | −0.048917939 | −0.170921593 | 0.297024198 | −0.077184667 |
| ITPR1 | 0.183411408 | 0.083381485 | −0.141270423 | −0.12552247 |
| KHDRBS3 | −0.564530854 | −0.17927316 | 0.456591544 | 0.28721247 |
| KL | 0.197687519 | 0.074877357 | −0.118292545 | −0.154272331 |
| KLHL5 | −0.422836082 | −0.229746693 | 0.318094341 | 0.334488433 |
| KLK12 | −0.029242768 | −0.008884391 | 0.04635967 | −0.008232511 |
| KRT14 | −0.245733109 | −0.267814551 | 0.345582498 | 0.167965162 |
| LAMP5 | −0.368387835 | −0.273814749 | 0.462626597 | 0.179575987 |
| LGR5 | −0.022611266 | 0.04849323 | −0.113626046 | 0.087744082 |
| LINC00668 | 0.097144338 | 0.050642793 | −0.031381451 | −0.116405681 |
| LRG1 | −0.485409227 | −0.022718861 | 0.23467065 | 0.273457438 |
| LRRC9 | 0.016206927 | 0.318087151 | −0.204867406 | −0.129426672 |
| LY6K | 0.191476228 | 0.050023676 | −0.075708279 | −0.165791625 |
| MAOB | −0.121394451 | 0.079907849 | 0.05812382 | −0.016637218 |
| MCM7 | −0.444444877 | −0.094559844 | 0.08595411 | 0.453050611 |
| MEIS1 | −0.185348904 | −0.055959282 | 0.251628339 | −0.010320154 |
| MEX3A | −0.264902151 | −0.757419417 | 0.075760473 | 0.946561095 |
| MFAP5 | −0.225308541 | −0.002197546 | 0.186776718 | 0.040729369 |
| MKRN1 | 0.522667907 | 0.483899696 | −0.670041149 | −0.336526454 |
| MORC2 | −0.631046304 | 0.45864546 | −0.148819026 | 0.32121987 |
| MUC1 | −0.026483669 | −0.052114392 | −0.000292394 | 0.078890455 |
| MUC12 | −0.100969107 | −0.002949073 | 0.087217317 | 0.016700863 |
| MUC15 | −0.084912519 | −0.086560339 | 0.10789439 | 0.063578468 |
| MUC16 | −0.594650223 | −0.161803024 | 0.349611136 | 0.406842111 |
| MUC2 | 0.150975319 | 0.081666806 | −0.035721253 | −0.196920873 |
| MYL2 | 0.012177962 | −0.036018491 | 0.034070451 | −0.010229922 |
| MYOC | −0.196326158 | 0.053766172 | 0.12916928 | 0.013390707 |
| MYOM1 | −0.6586284 | 0.077781758 | 0.312244662 | 0.424165497 |
| NPM1 | −0.392852375 | 0.092460322 | 0.199171595 | 0.101220459 |
| NPY4R | 0.158375867 | 0.175449654 | −0.120883465 | −0.212942056 |
| NRK | −0.004227984 | −0.015533393 | −0.068368783 | 0.08813016 |
| ODC1 | 0.06960318 | 0.133742032 | −0.083084328 | −0.120260885 |
| ONECUT2 | −0.624547292 | 0.084094951 | 0.331610357 | 0.208841984 |
| OPTN | −0.660307934 | −0.357920792 | 0.875836046 | 0.14239268 |
| OR2L13 | 0.096377647 | 0.096967288 | 0.001488876 | −0.000899235 |
| OR51F1 | 0.179965551 | 0.140772579 | −0.134607882 | −0.186130248 |
| OR52N5 | 0.065147115 | −0.094151256 | −0.072584978 | 0.101589119 |
| ORM1 | 0.01226212 | 0.028736593 | −0.013857749 | −0.027140964 |
| OS9 | 0.955446802 | −0.150484788 | −0.301263792 | −0.503698223 |
| OXCT1 | −0.111911957 | 0.140004069 | −0.103706499 | 0.075614386 |
| P2RY14 | −0.125777806 | 0.012565672 | 0.208566486 | −0.095354352 |
| PABPC3 | 0.208684832 | 0.111708877 | −0.20460415 | −0.115789559 |
| PARG | −0.126508019 | 0.313379267 | −0.032965615 | −0.153905633 |
| PCDHA12 | 0.017458235 | 0.213614081 | −0.14681968 | −0.084252636 |
| PDAP1 | 0.2185068 | 0.095597033 | −0.040000266 | −0.274103567 |
| PDE11A | 0.129334288 | 0.13586068 | −0.042896586 | −0.222298382 |
| PICK1 | 0.840902287 | 0.339779439 | −0.574647662 | −0.606034064 |
| PIGR | 0.003160155 | −0.166063356 | 0.1001536 | 0.062749601 |
| PLCXD3 | −0.125484555 | −0.347518256 | 0.153426515 | 0.319576296 |
| PLEK | −0.626048141 | −1.069776278 | 1.603152282 | 0.092672136 |
| PMM2 | −0.044700744 | −0.112408797 | −0.118604025 | 0.275713565 |
| PNMA1 | 0.020320771 | 0.027884794 | 0.20732526 | −0.255530824 |
| PODXL2 | 0.594059223 | 0.505709675 | −0.491819407 | −0.607949491 |
| POTEB3 | 0.197319664 | 0.137335087 | −0.159118349 | −0.175536402 |
| POTEC | 0.185184127 | 0.127631445 | −0.106186111 | −0.20662946 |
| POTED | 0.057391048 | 0.104091316 | −0.092970964 | −0.0685114 |
| PPP1R14B | 0.051323993 | −0.259878785 | −0.191229545 | 0.399784337 |
| PPP1R18 | 0.065377047 | −0.438295824 | 0.494612727 | −0.12169395 |
| PRKCB | −0.367833247 | 0.179337405 | 0.809388614 | −0.262217963 |
| PRKG2 | 0.172444286 | 0.05508838 | −0.047897287 | −0.179635378 |
| PYHIN1 | −0.069056278 | −0.874408499 | 1.352864335 | −0.409399557 |
| RAP1A | −0.138749163 | 0.128357481 | 0.121389385 | −0.110997703 |
| REXO2 | −0.000578202 | 0.042612354 | −0.000196848 | −0.041837304 |
| RIMS1 | 0.671651716 | 0.512793185 | −0.841709936 | −0.342734965 |
| RLIM | −0.146356465 | 0.240326341 | −0.021453318 | −0.072516558 |
| RND3 | −0.139953454 | −0.091754352 | 0.152953994 | 0.078753812 |
| RNPS1 | 0.6450799 | 0.941753034 | −0.732953681 | −0.853879253 |
| RPAIN | 0.011201182 | 0.361027297 | −0.273031913 | −0.099196566 |
| RPL14 | −0.012908321 | 0.039758679 | −0.110081609 | 0.083231252 |
| RPL22 | 0.528254056 | −0.182425702 | −0.218358561 | −0.127469793 |
| RPS3 | 0.014995222 | 0.078583821 | 0.034501401 | −0.128080444 |
| RRM2 | −3.376224643 | 1.888467967 | 0.409139813 | 1.078616864 |
| RSPO3 | 0.24906186 | 0.004969561 | −0.136310699 | −0.117720722 |
| S100A2 | −0.134786109 | −0.510653544 | 0.220366684 | 0.42507297 |
| S100A9 | −0.181046052 | −0.120016399 | 0.502252217 | −0.201189765 |
| S100P | −0.133256857 | −0.056469071 | 0.138772543 | 0.050953385 |
| SCUBE2 | −0.088337831 | −0.023438788 | 0.188428756 | −0.076652137 |
| SERPINB5 | −0.138395574 | 0.001604685 | 0.055673814 | 0.081117076 |
| SERTM1 | −0.081822378 | −0.358834749 | 0.066033683 | 0.374623444 |
| SET | −0.149698146 | −0.136209948 | −0.015434621 | 0.301342715 |
| SKA3 | −2.742077086 | 1.034631613 | 0.941985421 | 0.765460053 |
| SLC39A7 | 0.433400026 | 0.353767162 | −0.393314597 | −0.393852591 |
| SLC44A5 | −0.04393085 | −0.026411776 | 0.024489488 | 0.045853137 |
| SLC5A4 | 0.134749285 | 0.12446627 | −0.104440925 | −0.154774629 |
| SLFN11 | −1.121004516 | 0.056562259 | 0.811036306 | 0.253405951 |
| SMS | 0.150122269 | 0.193047326 | −0.17935136 | −0.163818234 |
| SNAP91 | 0.19437077 | 0.162488305 | −0.148722806 | −0.208136269 |
| SPINK1 | −0.047286456 | 0.038605157 | 0.053772693 | −0.045091394 |
| SRPX2 | −0.189724153 | 0.27563281 | 0.087772368 | −0.173681025 |
| SSRP1 | −1.063979726 | 0.478342832 | 0.594807595 | −0.009170702 |
| STAC | 0.092639837 | −0.33078977 | −0.144625529 | 0.382775462 |
| STAU2-AS1 | 0.235503772 | 0.002564999 | −0.346281021 | 0.10821225 |
| SUPT5H | −0.107420947 | 0.159298731 | −0.075811132 | 0.023933348 |
| SYCE1 | −0.041813432 | −0.06942789 | −0.068529586 | 0.179770908 |
| SYT6 | 0.398148582 | −0.30320119 | −0.099300717 | 0.004353325 |
| TBC1D3B | 0.03003626 | 0.13734471 | −0.122619646 | −0.044761324 |
| TBX4 | 0.002849532 | −0.04010964 | 0.159216065 | −0.121955956 |
| THBS4 | 0.029143205 | −0.16874665 | 0.201836651 | −0.062233206 |
| TLR1 | −0.193485483 | −0.235394646 | 1.048625251 | −0.619745123 |
| TNNI1 | 0.300532294 | −0.258966519 | 0.004504824 | −0.0460706 |
| TPM2 | 0.193503622 | −0.226843092 | −0.084064134 | 0.117403604 |
| TSEN15 | 0.559969512 | 0.30534883 | −0.324946434 | −0.540371908 |
| TSPYL2 | 0.257210537 | −0.187052223 | 0.156389065 | −0.226547379 |
| TUBB4A | 0.010911814 | −0.045443422 | −0.021695083 | 0.056226692 |
| TUFM | 0.186899994 | 0.119247177 | −0.194759233 | −0.111387938 |
| UGDH | 0.041191314 | 0.030464964 | −0.050847835 | −0.020808443 |
| UGT2B4 | 0.071600533 | −0.065002717 | −0.009471834 | 0.002874018 |
| UPK1A | −0.048103941 | −0.004892419 | −0.010559871 | 0.06355623 |
| UQCRH | −0.339868013 | −0.045079892 | 0.078968014 | 0.30597989 |
| USP17L10 | −0.033698121 | −0.038855677 | −0.011977257 | 0.084531054 |
| VCAM1 | −0.282169839 | −0.503498508 | 0.726852712 | 0.058815635 |
| VSTM2L | 0.031038009 | −0.023484095 | 0.097606178 | −0.105160091 |
| VTCN1 | −0.354472048 | −0.378544671 | 0.107686335 | 0.625330385 |
| VWA2 | 0.270324256 | 0.08413516 | −0.315572978 | −0.038886437 |
| WDR93 | 0.478249924 | 0.321588882 | −0.481266493 | −0.318572313 |
| WIF1 | −0.143129064 | −0.001396888 | 0.139106117 | 0.005419835 |
| WISP1 | −0.232443275 | −0.202433733 | 0.486205307 | −0.051328299 |
| ZC3H13 | −0.25689507 | 0.038157716 | 0.163128365 | 0.055608989 |
| ZCCHC13 | 0.202233838 | −0.038738017 | −0.28460471 | 0.121108888 |
| ZEB2 | −0.503881919 | −0.332908571 | 0.865650415 | −0.028859926 |
| ZNF556 | −0.261246764 | −0.066214389 | 0.121002112 | 0.20645904 |
| ZNF608 | 0.378032265 | 0.061702629 | −0.356109727 | −0.083625167 |

### Subsets of Table 2

Tables 2.1-2.10 above list subsets of targets of those listed in Table 2 which can be used in the embodiments disclosed herein. Tables 2.1-2.10 list the accuracy of classifying samples as LD, LP, BI or BN as compared to using all 215 targets listed in Tables 2. That is to say that if the 20 targets listed in Table 2.1 are used, 85% of the samples will be assigned the same subtype had all 215 targets listed in Tables 2 been used to subtype the sample. If the additional 10 targets listed in Table 2.2 are added to those listed in Table 2.1 (a total of 30 targets), 88% of the samples will be assigned the same subtype had all 215 targets listed in Table 2 been used. As expected, the more targets used, the greater the consistency between the subset of targets listed in Tables 2.1-2.10 and the results obtained using all 215 targets listed in Table 2. Table 2.11 lists all the targets listed in Table 2, and thus there is 100% agreement between using those targets in Table 2.11 and Table 2.

### Statistical Analysis

In the demographics tables, ANOVA and Chi-squared test were used to evaluate differences between continuous and categorical variables, respectively, between patient groups. Gleason score was stratified into low (<7), intermediate (7), and high risk (8-10). PSA was stratified into low (<10 ng/mL), intermediate (10-20 ng/mL), and high risk (>20 ng/mL) in a similar manner. SM, ECE, SVI, and LNI were considered as binary variables. Cox regression was used for both univariable and multivariable analysis (UVA/MVA). Stratification by cohort was used when performing UVA/MVA analyses to account for baseline differences between cohorts. The interaction term for treatment and subtype in a Cox model was used to evaluate prediction of treatment response, and a significant interaction Wald test p-value indicated that a subtype could predict response to a particular drug or treatment. To apply the PSC model on archival samples collected >5 years ago, quantile matching was applied to adjust the distribution of expression on the samples. Statistical significance was set as a two-tailed p-value <0.05. All statistical analyses were performed in R 4.0.2.

PSC Subtypes Association with Treatment and Outcomes Data.

To investigate whether PSC subtypes were associated with long-term survival differences, subtypes were examined in a cohort of 855 prostate cancer patients treated with radical prostatectomy previously described by Spratt et al., JCO 2018. The primary clinical endpoint was distant metastasis-free survival (DMFS), with secondary clinical endpoint of overall survival (OS). All endpoints were defined from time of surgery until time of the event, death, or last follow-up. A second cohort of NCCN high risk men was also used to examine survival differences between the subtypes in patients treated with primary radiotherapy.

### Cohort for Predicting Response to ADT Duration (TS006).

To investigate if subtypes could predict response to duration of hormone therapy, samples from 265 NCCN high risk men from three Phase 3 randomized trials (RTOG 92-02, 94-13 & 99-02) treated with primary radiotherapy with short (4 months) or long (28 months) duration of adjuvant ADT (Nguyen et al., ASTRO 2021) were used. Luminal subtype tumors (LD & LP) were grouped to compare to basal subtype tumors (BI & BN).

### Cohort for Predicting Response to Radiation Therapy (RTOG 01-26).

To investigate if subtypes could predict response to radiotherapy dose, a cohort of 215 men treated with standard dose (70 Gy) as compared to dose-escalation (79 Gy) in the Phase 3 RTOG 01-26 randomized trial of NCCN intermediate risk men (Spratt et al., ASCO GU 2022) were examined. Luminal subtype tumors (LD & LP) were grouped to compare to basal subtype tumors (BI & BN).

### Cohort for Predicting Response to Addition of ADT to Salvage Radiotherapy (RTOG 96-01)

The investigate if subtypes could predict response to addition of hormone therapy to radiotherapy the model in 352 men treated with salvage radiotherapy after radical prostatectomy with or without 24 months of ADT in the Phase 3 RTOG 96-01 randomized trial of radical prostatectomy patients were examined (Feng et al., JAMA Onc 2021). Luminal subtype tumors (LD & LP) were grouped to compare to basal subtype tumors (BI & BN).

### Cohort for Predicting Response to Chemotherapy (ECOG 3805).

To investigate if subtypes could predict chemotherapy response, 160 samples from the CHAARTED (ECOG 3805) randomized controlled clinical trial were examined (Hamid et al., Annals of Oncology 2021). Patients with metastatic hormone sensitive prostate cancer were randomized to either ADT alone or ADT plus docetaxel. Luminal proliferating (LP) were compared to non-LP (LD, BI & BN) tumors.

### Results

To subtype prostate cancer, 32,000 patient tumor transcriptomes were retrieved and grouped based on expression patterns of a curated panel of 21 seed features (Table 1). Hierarchical clustering revealed four main expression patterns. To refine the classification and to create a single sample classifier that could be used to classify an individual sample without utilization of a centroid or reference population (a limitation of models such as the PAM50 classifier), multinomial logistic regression was used to select for genes that could be used to reproduce the initial four ‘seed’ clusters. Several gene filters were applied, and after pairwise comparisons between samples in the clusters, a total of 215 genes with non-zero coefficients were selected in the final clustering solution (Table 2).

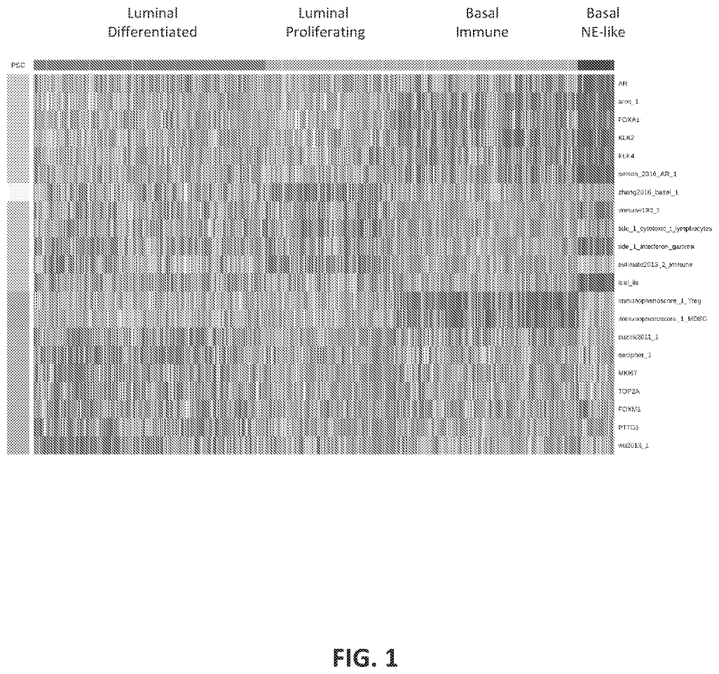

The final clustering solution further refined the PAM50 subtyping model, splitting the basal tumors further into two subtypes. We named the four clusters as Luminal Differentiated (LD), Luminal Proliferating (LP), Basal Immune (BI) and Basal Neuroendocrine-like (BN). FIG. 1 shows the 21 seeding features used in the PSC model to cluster patient samples in the training and testing cohorts (n=64,000).

### Biology of Luminal Differentiated (LD) Tumors.

Luminal Differentiated (LD) tumors are characterized by the highest expression of prostate terminal differentiation markers such as KLK2, KLK3 and KLK4 as well as the highest levels of the hallmarks of cancer (Liberzon et al. Cell Reports 2017) androgen response signature, which includes genes that define biological response to androgen hormones such as testosterone. These tumors had the lowest levels of proliferation markers such as NUSAP1, PTTG1, TOP2A and UBE2C. LD tumors were predominately classified by the PAM50 model as luminal A subtype tumors.

### Biology of Luminal Proliferating (LP) Tumors.

Luminal Proliferating (LP) tumors had the highest expression of the androgen receptor (AR) gene, downstream targets of the AR transcriptional activity such as AMACR, FOLH1 (PSMA), KRT8 and TMPRSS2 as well as previously reported AR activity signatures (Kumar 2016 and Spratt 2019). Transcription factors associated with luminal biology such as GATA2, FOXA1 and NKX3-1 were highest in these tumors. The hallmarks cholesterol metabolism and glycolysis were highest in these tumors as were pathways associated with PI3K, mTOR signaling and a pTEN deletion signature (Saal 2007). LP also had the highest expression of the hallmark unfolded protein response signature and the ubiquitin-mediated proteasome gene UBE2C. Proliferation markers such as CCND1, FOXM1, MKI67, NUSAP1, TOP2A, TPX2, hallmarks of cancer associated with regulation of the cell cycle and proliferation such as E2F, MYC targets v2, G2M, mitotic spindle and other previously reported proliferation signatures such as Cuzick 2011 (Cuzick et al., Lancet Oncology 2011) and Cheville 2008 (Cheville et al., JCO 2008) were highest in the LP subtype. Genes such as EZH2, RAD21, PLK1, TP53 and signatures involved in DNA damage and repair were at their highest levels in this subtype. The hallmark of cancer DNA repair signature, TP53 pathway signature (Chipidza et al., 2020 Clin Genitourin. Cancer), chromosomal instability signature CIN70 (Carter et al., Nat Genet. 2006), and DNA damage repair deficiency signatures (Knijnenburg et al., 2018 Cell Rep) such as those developed for Fanconi Anemia, Non-homologous end joining, Base excision repair and Homologous recombination were also highest in this subtype as is the homologous recombination deficiency (HRD) signature (Weiner et al., 2020) that has been shown to predict the presence of functional BRCA2 mutations. LP tumors were predominately classified by the PAM50 model as luminal B subtype tumors.

### Biolog of Basal Immune (BI) Tumors.

Basal Immune (BI) tumors had the highest expression of non-AR sex steroid transcription factors such as the estrogen receptor 1 (ESR1), glucocorticoid receptor (NR3C1) and progesterone receptor (PGR). BI also had lower scores of the ADT response signature (ADT-RS, Karnes CCR 2019). The basal lineage CD49f signature (Smith et al. Proc Natl Acad Sci USA. 2015; 112(47):E6544-6552) is increased in BI, as are basal differentiation markers such as KRT5 and TP63. BI had the highest expression of CHD1 and pTEN. BI subtype tumors had highest expression of angiogenesis, apoptosis, epithelial to mesecnchymal (EMT), hypoxia and kRas signaling up but lowest expression of the kRas signaling down and DNA repair hallmark of cancer signatures. In addition, BI had the highest expression of the IL2-JAK-STAT5, IL6-JAK-STAT3, TNFA, TGF beta, inflammatory response, interferon alpha and gamma tumor hallmark of cancer signatures. BI tumors had the highest levels of bulk immune infiltrate as measured by the immune 190 signature (Seiler et al., Clin Can Res 2019). When considering other tumor immune microenvironment signatures developed to predict response to immune checkpoint blockade such as the Tumor Immune Dysfunction and Exclusion modules (Jiang et al., Nat Med 2018) and Immunophenoscores (Charoentong et al., Cell Reps 2017), BI tumors had the highest levels immune effector cells (eg CD8+ and CD4+ T cells), the lowest levels of immune suppressive cell signatures (eg T reg, myeloid derived suppressor cells) and M2-to-M1 macrophage ratio. Interestingly, the LP and BN tumors had the highest levels of these tumor immune suppressive microenvironment signatures.

### Biology of Basal-Neuroendocrine-Like (BN) Tumors.

Basal tumors are characterized by the lowest expression of the AR gene, terminal differentiation markers such as KLK2, KLK3 and KLK4 as well as AR transcriptional activity signatures. Basal-Neuroendocrine-like (BN) tumors had the lowest levels of CCND1, CHD1, RBI, SPOP and TP53 gene expression. BN tumors had the highest levels of the p53 mutation gene expression signatures (Donehower 2019, Chipidza 2020) and the pRB loss signature (Chen CCR 2019). Basal tumors also had the highest levels of the PAM50 basal signature, the hallmark of cancer reactive oxygen species, kRas signaling down, MYC targets v1 and oxidative phosphorylation signatures. Basal tumors had the highest expression of neuroendocrine and small cell markers such as DCX, SYP, TUBB2B as well as neuroendocrine subtype gene expression signatures such as those reported in Balanis et al., (Cancer Cell 2019), Beltran et al., (Nat Med 2016), Kumar et al., (Nat Med 2016) and Tsai et al., (BMC Ca 2017).

### PSC Subtypes are Prognostic.

A panel of 21 previously developed prognostic gene expression signatures was examined. Overall, LD tumors had the lowest prognostic signature scores but for the other subtypes displayed some heterogeneity. For example, LP tumors had the highest levels for most of the prognostic signatures ( 13/21) including Bibikova 2007, Bismar 2006, Cheville 2008, Cuzick 2011, Glinsky 2005, Long 2011, Ramaswamy 2003, Singh 2002, Stephenson 2005, Talantov 2010, Varambally 2005, Wu 2013. Agell 2012, Nakagawa 2008 were prognostic signatures which had highest levels in the BI tumors, whereas in Basal Neuroendocrine-like tumors Decipher, Lapointe 2004, Penney 2011, Yu 2007 and Larkin 2012 had the highest levels.

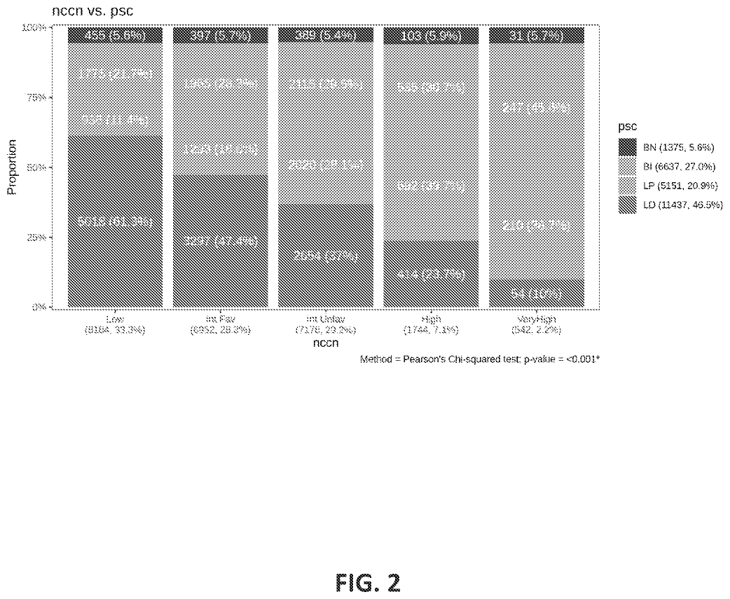

For patients at initial diagnosis, we assessed whether subtypes were associated with differences in baseline clinicopathologic variables. For example, LD tumors which were enriched among younger men (median age at diagnosis 66 years old), had lower PSA levels at diagnosis, more lower stage (<cT1c) and more lower grade disease (56% Grade Group 1 were found in this subtype) (Table 3). Conversely, higher risk clinicopathologic risk factors were enriched in the LP and BI subtypes. In particular BI tumors had more high-grade disease at diagnosis (46% of Grade Group 5 were found in this subtype). BI also had more higher stage disease (≥cT3). Overall, PSC was found to further stratify clinical risk models used at diagnosis for risk stratification purposes (FIG. 2). Amongst NCCN low risk men, LD was the most common subtype (61%) whereas among men with NCCN very high risk, BI was the most common subtype (46%). Similarly, after radical prostatectomy more high-grade disease was found in the LP and BI subtypes as compared to LD. Notably, BI had the highest frequency of pT3 disease and lymph node invasion suggesting tumor biology of this subtype (i.e., highly immune infiltrated) may portend a greater ability to spread to seminal vesicles (pT3b) and lymph nodes as compared to the other subtypes (Table 4).

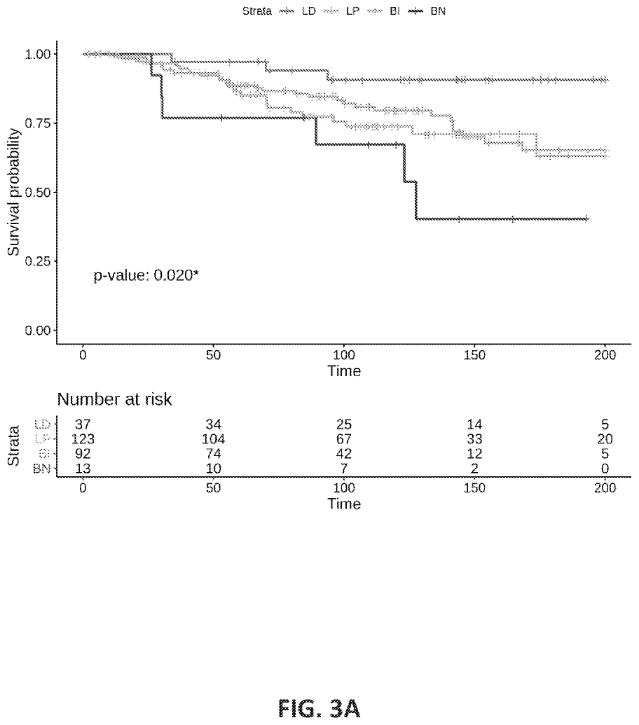

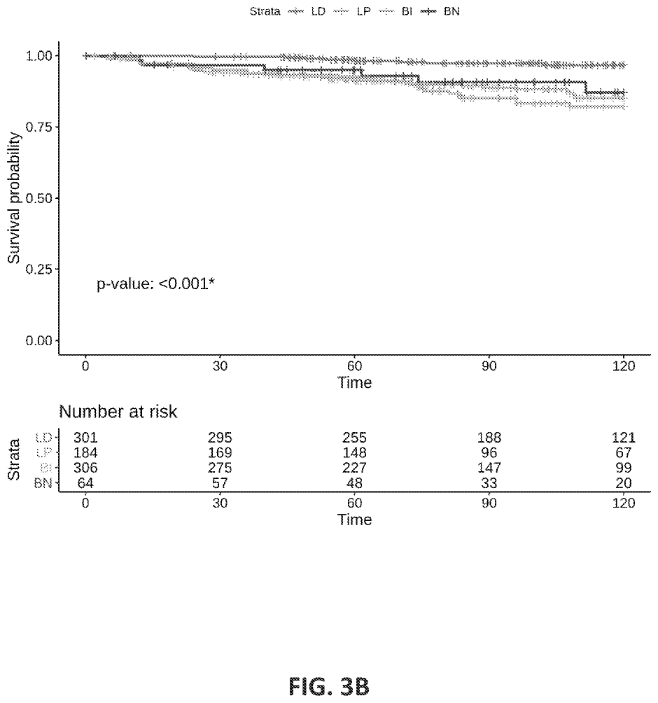

Associations of the subtypes was examined with long-term clinical outcomes in patients treated with radiotherapy (RT) and radical prostatectomy. Luminal Differentiated patients consistently have significantly better outcomes for all endpoints compared to Luminal Proliferating, Basal Immune and Basal NE-like subtypes. The RT cohort (TS-006), which had predominately high risk non-metastatic disease the 10-year actuarial rates for DMFS were, 91% for LD compared to 80a, 74% and 67 for LP, BI and BN; and for OS, 69% for LD compared to 56%, 49% and 46% for LP, BI and BN tumors (FIG. 3A).

TABLE 3

| | Value | LD: 14095 | LP: 6219 | BI: 7793 | BN: 1627 | P | |
|---|---|---|---|---|---|---|---|
| PSC | Category | (47.40) | (20.92) | (26.21) | (5.47) | Value | Total |
| age_bx | 1st Qu. | 60 | 63 | 63 | 63 | <0.001* | 62 |
| | Median | 66 | 69 | 68 | 68 | | 67 |
| | 3rd Qu. | 71 | 74 | 73 | 73 | | 72 |
| | NA | 1214 | 549 | 773 | 170 | | 2706 |
| | Count | | | | | | |
| African | 0 | 13477 | 5977 | 7427 | 1561 | 0.053 | 28442 |
| American | | (47.38) | (21.01) | (26.11) | (5.49) | | (95.65) |
| | 1 | 379 | 126 | 218 | 43 | | 766 |
| | | (49.48) | (16.45) | (28.46) | (5.61) | | (2.58) |
| | NA | 239 | 116 | 148 | 23 | | 526 |
| | | (45.44) | (22.05) | (28.14) | (4.37) | | (1.77) |
| Family | 0 | 13401 | 5940 | 7497 | 1580 | <0.001* | 28418 |
| history | | (47.16) | (20.90) | (26.38) | (5.56) | | (95.57) |
| | 1 | 455 | 163 | 148 | 24 | | 790 |
| | | (57.59) | (20.63) | (18.73) | (3.04) | | (2.66) |
| | NA | 239 | 116 | 148 | 23 | | 526 |
| | | (45.44) | (22.05) | (28.14) | (4.37) | | (1.77) |
| Clings p | 3 | 12497 | 4088 | 5822 | 1302 | <0.001* | 23709 |
| | | (52.71) | (17.24) | (24.56) | (5.49) | | (79.74) |
| | 4 | 1389 | 1971 | 1743 | 293 | | 5396 |
| | | (25.74) | (36.53) | (32.30) | (5.43) | | (18.15) |
| | 5 | 25 | 68 | 121 | 14 | | 228 |
| | | (10.96) | (29.82) | (53.07) | (6.14) | | (0.77) |
| | NA | 184 | 92 | 107 | 18 | | 401 |
| | | (45.89) | (22.94) | (26.68) | (4.49) | | (1.35) |
| Clings s | 3 | 8200 | 2656 | 3807 | 823 | <0.001* | 15486 |
| | | (52.95) | (17.15) | (24.58) | (5.31) | | (52.08) |
| | 4 | 5628 | 3263 | 3583 | 750 | | 13224 |
| | | (42.56) | (24.67) | (27.09) | (5.67) | | (44.47) |
| | 5 | 83 | 208 | 296 | 36 | | 623 |
| | | (13.32) | (33.39) | (47.51) | (5.78) | | (2.10) |
| | NA | 184 | 92 | 107 | 18 | | 401 |
| | | (45.89) | (22.94) | (26.68) | (4.49) | | (1.35) |
| Gleason | 1 | 7053 | 1362 | 2539 | 611 | <0.001* | 11565 |
| group | | (60.99) | (11.78) | (21.95) | (5.28) | | (38.89) |
| | 2 | 5431 | 2719 | 3248 | 688 | | 12086 |
| | | (44.94) | (22.50) | (26.87) | (5.69) | | (40.65) |
| | 3 | 1144 | 1291 | 1258 | 212 | | 3905 |
| | | (29.30) | (33.06) | (32.22) | (5.43) | | (13.13) |
| | 4 | 196 | 506 | 311 | 57 | | 1070 |
| | | (18.32) | (47.29) | (29.07) | (5.33) | | (3.60) |
| | 5 | 87 | 249 | 330 | 41 | | 707 |
| | | (12.31) | (35.22) | (46.68) | (5.80) | | (2.38) |
| C stage | T1a | 204 | 66 | 89 | 23 | <0.001* | 382 |
| | | (53.40) | (17.28) | (23.30) | (6.02) | | (1.28) |
| | T1b | 56 | 15 | 16 | 3 | | 90 |
| | | (62.22) | (16.67) | (17.78) | (3.33) | | (0.30) |
| | T1c | 9675 | 3784 | 5279 | 1140 | | 19878 |
| | | (48.67) | (19.04) | (26.56) | (5.73) | | (66.85) |
| | T2 | 981 | 603 | 582 | 104 | | 2270 |
| | | (43.22) | (26.56) | (25.64) | (4.58) | | (7.63) |
| | T2b | 243 | 228 | 233 | 40 | | 744 |
| | | (32.66) | (30.65) | (31.32) | (5.38) | | (2.50) |
| | T2c | 307 | 233 | 255 | 36 | | 831 |
| | | (36.94) | (28.04) | (30.69) | (4.33) | | (2.79) |
| | T3a | 21 | 32 | 35 | 8 | | 96 |
| | | (21.88) | (33.33) | (36.46) | (8.33) | | (0.32) |
| | T3b | 8 | 16 | 17 | 1 | | 42 |
| | | (19.05) | (38.10) | (40.48) | (2.38) | | (0.14) |
| | T4 | 0 | 5 | 7 | 0 | | 12 |
| | | (0.00) | (41.67) | (58.33) | (0.00) | | (0.04) |
| | NA | 2600 | 1237 | 1280 | 272 | | 5389 |
| | | (48.25) | (22.95) | (23.75) | (5.05) | | (18.12) |
| Bx percent | 1st Qu. | 0.167 | 0.17 | 0.167 | 0.167 | <0.001* | 0.167 |
| pos cores | | | | | | | |
| | Median | 0.25 | 0.333 | 0.286 | 0.286 | | 0.29 |
| | 3rd Qu | 0.429 | 0.5 | 0.5 | 0.5 | | 0.5 |
| | NA | 182 | 91 | 107 | 17 | | 397 |
| | Count | | | | | | |
| NCCN | Low | 5018 | 936 | 1775 | 455 | <0.001* | 8184 |
| | | (61.31) | (11.44) | (21.69) | (5.56) | | (27.52) |
| | Int. Fav | 3297 | 1293 | 1965 | 397 | | 6952 |
| | | (47.43) | (18.60) | (28.27) | (5.71) | | (23.38) |
| | Int. | 2654 | 2020 | 2115 | 389 | | 7178 |
| | Unfav | (36.97) | (28.14) | (29,47) | (5.42) | | (24.14) |
| | High | 414 | 692 | 535 | 103 | | 1744 |
| | | (23.74) | (39.68) | (30.68) | (5.91) | | (5.87) |
| | Very | 54 | 210 | 247 | 31 | | 542 |
| | High | (9.96) | (38.75) | (45.57) | (5.72) | | (1.82) |

TABLE 4

| | Value | LD: 11660 | LP: 7985 | BI: 12654 | BN: 2383 | | |
|---|---|---|---|---|---|---|---|
| PCS | Category | (33.62) | (23.02) | (36.49) | (6.87) | P Value | Total |
| age_rp | 1st Qu. | 58.6 | 61 | 60 | 60 | <0.001* | 60 |
| | Median | 64 | 66 | 65 | 66 | | 65 |
| | 3rd Qu. | 68.9 | 70 | 69 | 70 | | 69 |
| | NA | 772 | 306 | 424 | 47 | | 1549 |
| | Count | | | | | | |
| African | 0 | 8499 | 6514 | 10336 | 2135 | <0.001* | 27484 |
| American | | (30.92) | (23.70) | (37.61) | (7.77) | | (79.25) |
| | 1 | 367 | 162 | 489 | 72 | | 1090 |
| | | (33.67) | (14.86) | (44.86) | (6.61) | | (3.14) |
| | NA | 2794 | 1309 | 1829 | 176 | | 6108 |
| | | (45.74) | (21.43) | (29.94) | (2.88) | | (17.61) |
| Family | 0 | 8530 | 6463 | 10393 | 2105 | <0.001* | 27491 |
| history | | (31.03) | (23.51) | (37.81) | (7.66) | | (79.27) |
| | 1 | 325 | 203 | 407 | 98 | | 1033 |
| | | (31.46) | (19.65) | (39.40) | (9.49) | | (2.98) |
| | NA | 2805 | 1319 | 1854 | 180 | | 6158 |
| | | (45.55) | (21.42) | (30.11) | (2.92) | | (17.76) |
| Pathgs p | 3 | 7109 | 2733 | 5300 | 1038 | <0.001* | 16180 |
| | | (43.94) | (16.89) | (32.76) | (6.42) | | (46.65) |
| | 4 | 3674 | 4746 | 6418 | 1247 | | 16085 |
| | | (22.84) | (29.51) | (39.90) | (7.75) | | (46.38) |
| | 5 | 120 | 201 | 516 | 52 | | 889 |
| | | (13.50) | (22.61) | (58.04) | (5.85) | | (2.56) |
| | NA | 757 | 305 | 420 | 46 | | 1528 |
| | | (49.54) | (19.96) | (27.49) | (3.01) | | (4.41) |
| Pathgs s | 3 | 3725 | 3064 | 4196 | 833 | <0.001* | 11818 |
| | | (31.52) | (25.93) | (35.51) | (7.05) | | (34.08) |
| | 4 | 6514 | 3574 | 5747 | 1134 | | 16969 |
| | | (38.39) | (21.06) | (33.87) | (6.68) | | (48.93) |
| | 5 | 659 | 1041 | 2291 | 370 | | 4361 |
| | | (15.11) | (23.87) | (52.53) | (8.48) | | (12.57) |
| | NA | 762 | 306 | 420 | 46 | | 1534 |
| | | (49.67) | (19.95) | (27.38) | (3.00) | | (4.42) |
| Gleason | 1 | 1026 | 154 | 415 | 76 | <0.001* | 1671 |
| group | | (61.40) | (9.22) | (24.84) | (4.55) | | (4.82) |
| | 2 | 5956 | 2528 | 4649 | 926 | | 14059 |
| | | (42.36) | (17.98) | (33.07) | (6.59) | | (40.54) |
| | 3 | 2680 | 2887 | 3729 | 752 | | 10048 |
| | | (26.67) | (28.73) | (37.11) | (7.48) | | (28.97) |
| | 4 | 603 | 967 | 967 | 206 | | 2743 |
| | | (21.98) | (35.25) | (35.25) | (7.51) | | (7.91) |
| | 5 | 632 | 1143 | 2472 | 377 | | 4624 |
| | | (13.67) | (24.72) | (53.46) | (8.15) | | (13.33) |
| sm | 0 | 5067 | 3558 | 5662 | 1073 | <0.001* | 15360 |
| | | (32.99) | (23.16) | (36.86) | (6.99) | | (44.29) |
| | 1 | 5839 | 4123 | 6580 | 1265 | | 17807 |
| | | (32.79) | (23.15) | (36.95) | (7.10) | | (51.34) |
| | NA | 754 | 304 | 412 | 45 | | 1515 |
| | | (49.77) | (20.07) | (27.19) | (2.97) | | (4.37) |
| epe | 0 | 6004 | 2756 | 4386 | 854 | <0.001* | 14000 |
| | | (42.89) | (19.69) | (31.33) | (6.10) | | (40.37) |
| | 1 | 4891 | 4925 | 7853 | 1483 | | 19152 |
| | | (25.54) | (25.72) | (41.00) | (7.74) | | (55.22) |
| | NA | 765 | 304 | 415 | 46 | | 1530 |
| | | (50.00) | (19.87) | (27.12) | (3.01) | | (4.41) |
| svi | 0 | 9716 | 6276 | 8941 | 1809 | <0.001* | 26742 |
| | | (36.33) | (23.47) | (33.43) | (6.76) | | (77.11) |
| | 1 | 1186 | 1404 | 3295 | 528 | | 6413 |
| | | (18.49) | (21.89) | (51.38) | (8.23) | | (18.49) |
| | NA | 758 | 305 | 418 | 46 | | 1527 |
| | | (49.64) | (19.97) | (27.37) | (3.01) | | (4.40) |
| lni | 0 | 10579 | 7260 | 11343 | 2203 | <0.001* | 31385 |
| | | (33.71) | (23.13) | (36.14) | (7.02) | | (90.49) |
| | 1 | 239 | 399 | 862 | 127 | | 1627 |
| | | (14.69) | (24.52) | (52.98) | (7.81) | | (4.69) |
| | NA | 842 | 326 | 449 | 53 | | 1670 |
| | | (50.42) | (19.52) | (26.89) | (3.17) | | (4.82) |
| bcr | 0 | 9298 | 6639 | 10800 | 2002 | <0.001* | 28739 |
| | | 32.35) | (23.10) | (37.58) | (6.97) | | (82.86) |
| | 1 | 1270 | 879 | 1223 | 312 | | 3684 |
| | | (34.47) | (23.86) | (33.20) | (8.47) | | (10.62) |
| | NA | 1092 | 467 | 631 | 69 | | 2259 |
| | | (48.34) | (20.67) | (27.93) | (3.05) | | (6.51) |

The RP cohort (Spratt et al., JCO 2018), which consisted of men with adverse pathology after RP (positive surgical margins or ≥pT3 disease) the 10-year actuarial rates for DMFS, 97% for Luminal Differentiated compared to 80%, 85% and 87% for LP, BI and BN; and for OS, 87% for LD compared to 79%, 79% and 85% for LP, BI and BN (FIG. 3B).

### PSC Subtypes Predict Response to Radiation Therapy Dose

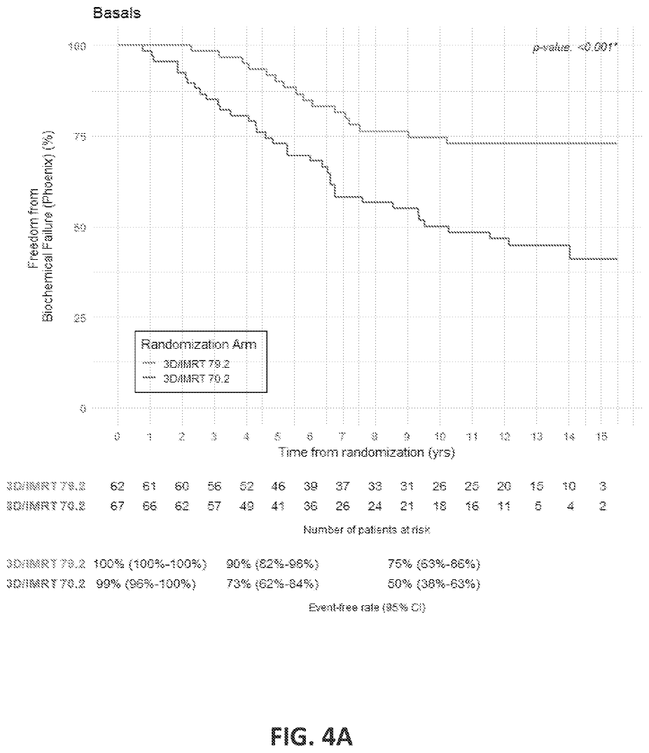

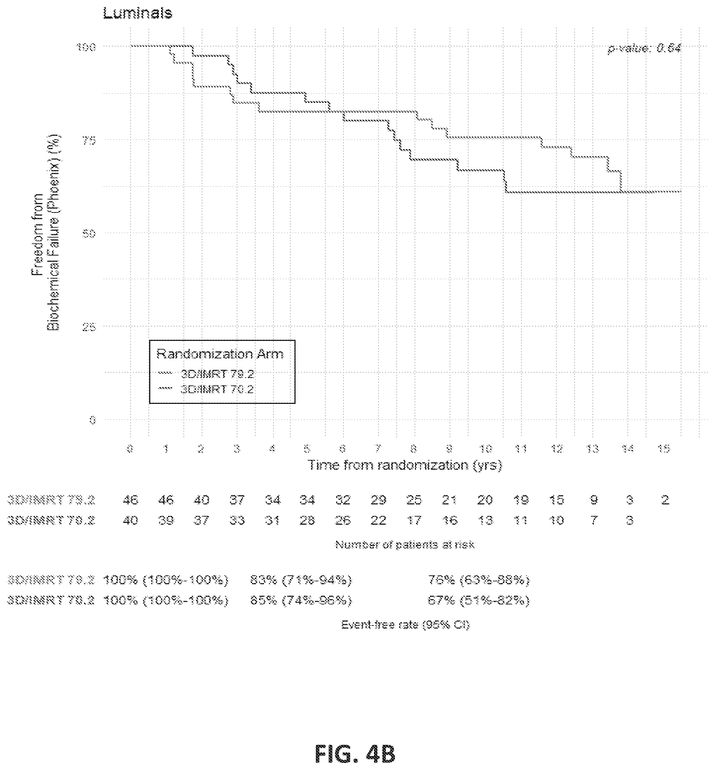

In a cohort of 215 intermediate risk men treated with two different doses of primary RT (70 Gy vs 79 Gy) from the RTOG 01-26 Phase 3 randomized controlled clinical trial we observed basal subtype tumors (BI & BN) to have improved outcomes with the dose-escalation (79 Gy) of radiotherapy. For example, at 10 years, 90% of basals that received dose-escalation (79 Gy) did not have biochemical recurrence as compared to 73% for those on the standard dose (70 Gy) (p<0.001, FIG. 4). In contrast, outcomes were not different between standard vs dose-escalation in those patients with luminal tumors (p=0.64). These results showed that prostate subtyping classifiers of the disclosure predict response to radiation therapy dose.

### PSC Subtypes Predict Response to Hormone Therapy Duration.

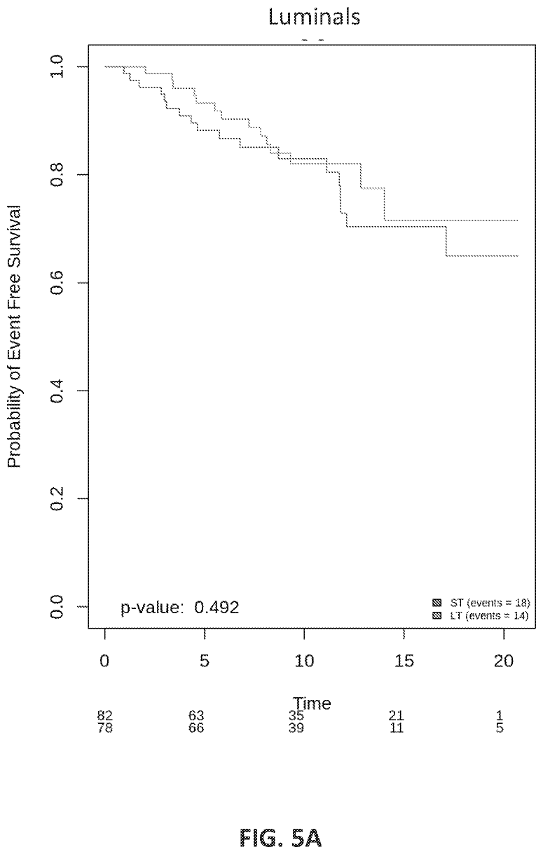

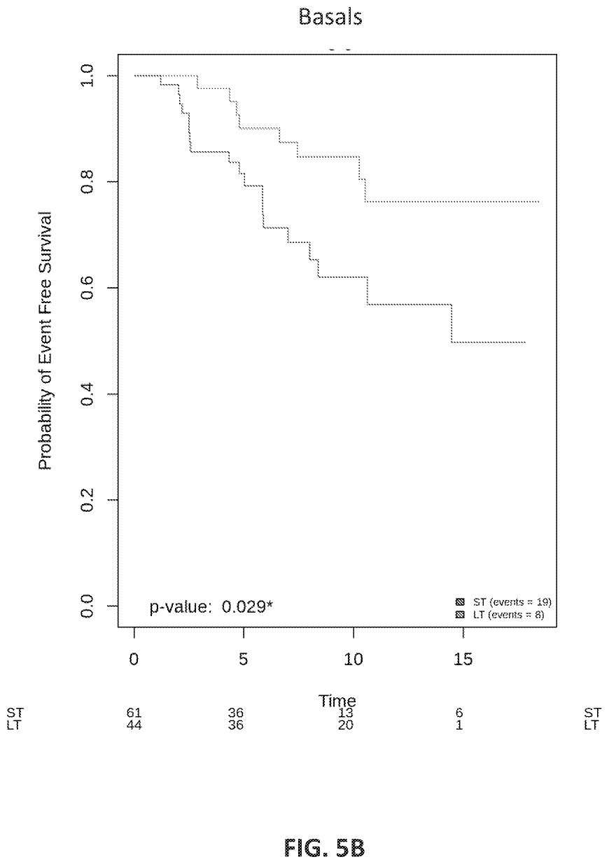

In a cohort of 265 high risk men randomized to treatment with primary RT (TS-006 study) with either 4 or 28 months of adjuvant ADT, basal subtype tumors (BI & BN) improved outcomes with long term ADT as compared to short term ADT. At 10 years, in basal tumors the distant metastasis-free survival was 85% for LT as compared to 62% for ST ADT (p<0.029, FIG. 5). No differences were observed in luminal tumors (p<0.5). These results showed that prostate subtyping classifiers of the disclosure predict response to hormone therapy duration.

### PSC Subtypes Predict Response to Hormone Therapy in the Salvage Setting.

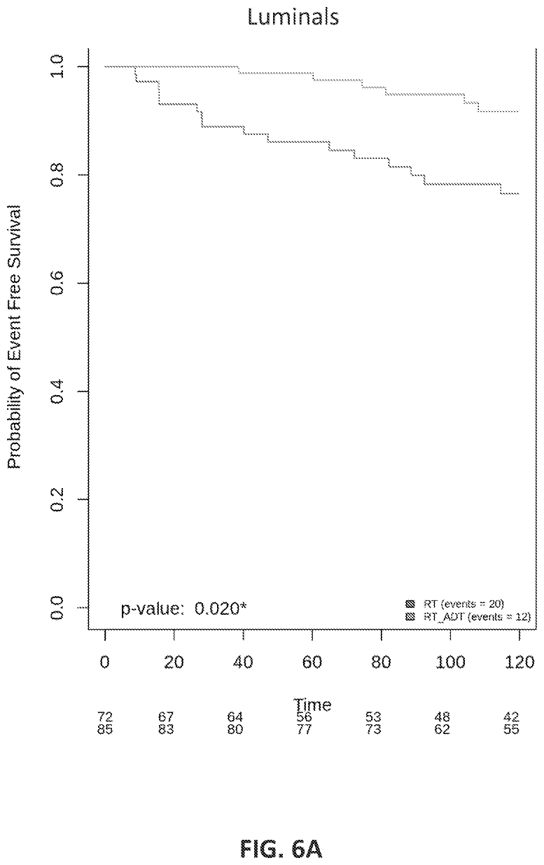

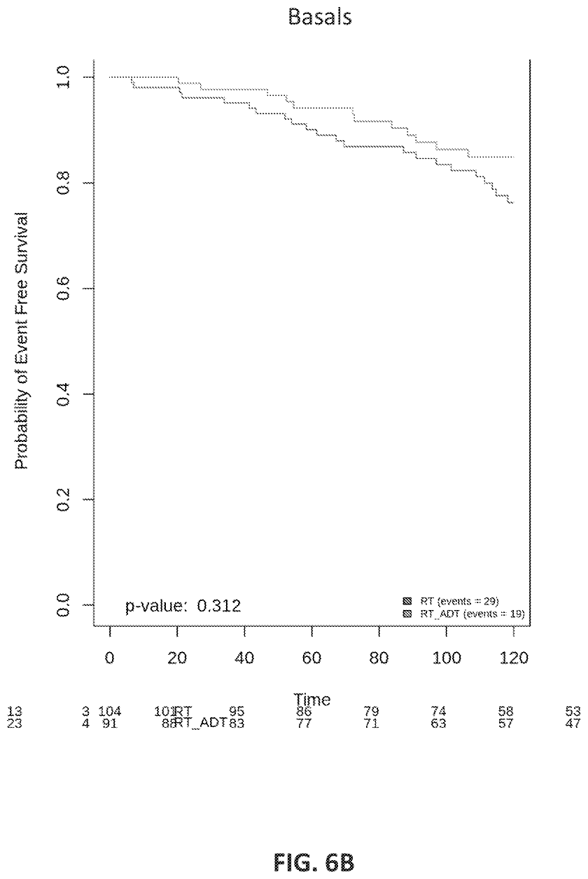

In a cohort of 352 RP patients that experienced biochemical recurrence, from the RTOG 96-01 Phase 3 randomized controlled clinical trial to receive salvage RT alone or in combination with 24 months of ADT, luminal subtype (LD & LP) tumors showed improved outcomes with the addition of ADT. At 10 years, in luminal tumors, the distant metastasis-free survival was 85% for RT+ADT as compared to 72% for RT alone (p<0.02, FIG. 6). In contrast, basal tumors had no improvement in outcomes with the addition of ADT (p=0.3). These results showed that prostate subtyping classifiers of the disclosure predict response to hormone therapy in the salvage setting.

PSC Subtypes Association with Predicted Response to Cancer Drugs.

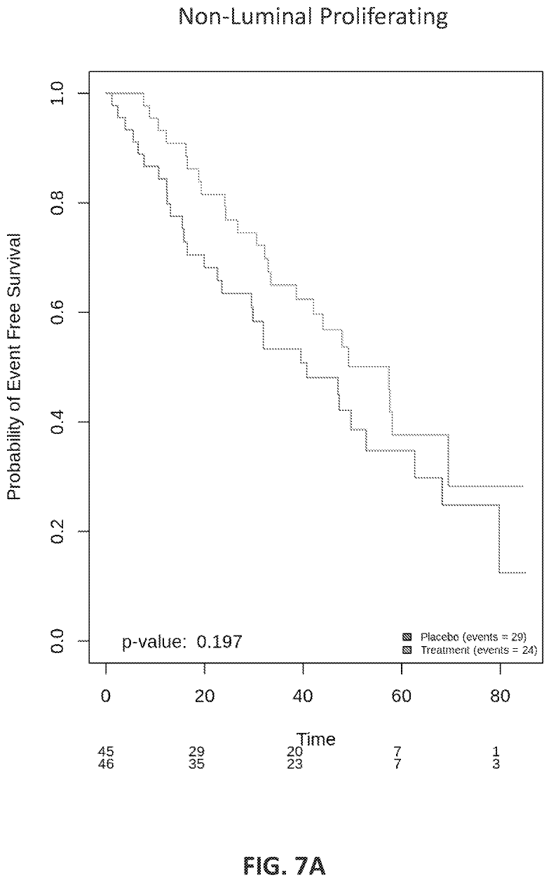

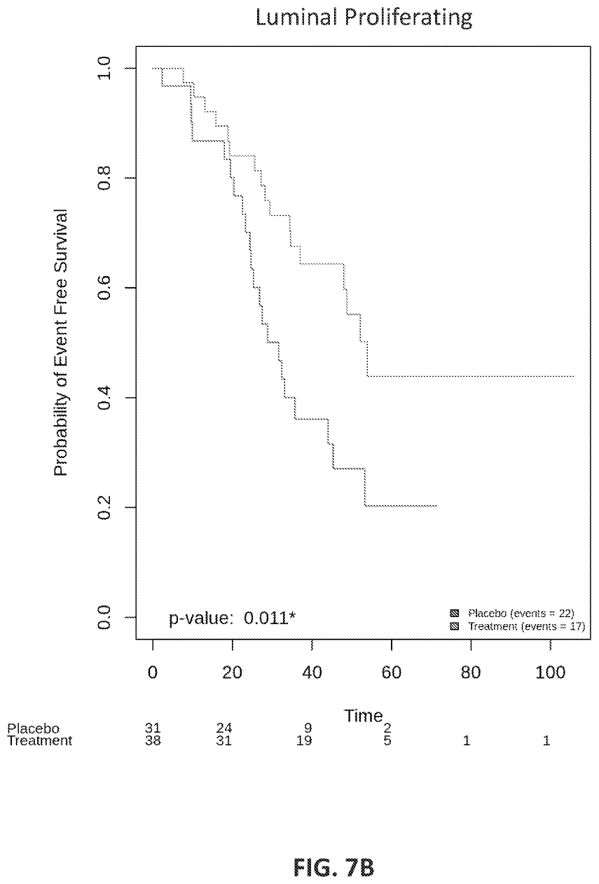

Drug sensitivity was calculated using in vitro drug sensitivity and microarray data to generate gene signatures predicting tumor sensitivity to 89 oncology drugs. No significant associations of predicted drug response signatures were observed with the LD subtype. Both luminal subtypes had the highest predicted response to abiraterone acetate, a commonly used hormonal therapy drug for prostate cancer. BN subtype tumors had the lowest levels for abiraterone. Drug response signatures that highly predicted responses for LP subtype included drugs that regulate the proteasome or cellular protein metabolism such as bortezomib, carfilzomib, alvespimycin and tanespimyicin as well as drugs that inhibit cellular division through abrogation of the microtubule complexes such as docetaxel and paclitaxel. An analysis of samples from the CHAARTED Phase 111 randomized clinical trial (ECOG 3805) was conducted that compared treatment of patients with newly diagnosed metastatic hormone sensitive prostate cancer with ADT alone or ADT+docetaxel chemotherapy. The LP subtype had significantly improved overall survival when treated with docetaxel in addition to ADT (FIG. 7). For example, at 5 years, overall survival in LP tumors was 44% in those treated with ADT+docetaxel as compared to 20% for ADT alone (p<0.011). For patients with non-LP tumors there was no difference in outcomes between the two arms (p=0.197). These results showed that prostate subtyping classifiers of the disclosure predict response to cancer drugs.

BI subtype tumors had the highest drug response predictions for protein kinase inhibitors such as dasatinib, erlotinib, gefitinib, ibrutinib, olaparib, pazopanib, vandetinib, staurosporine and hydroxy-staurosporine. In addition, BI subtype tumors had the highest predicted responses to mTOR pathway inhibitors such as rapamycin, everolimus as well as HMG CoA inhibitors such as lovostatin and somastatin. BN tumors had the highest drug response predictions for alkylating agents such as carboplatin, cisplatin, oxaliplatin, campothecin; topoisomerase inhibitors such as cyclosphosphamide, etoposide, ifosfamide, mitoxantrone, epirubicin, doxorubicin and vinca alkaloids such as vinorelbine, vincristine and vinblastine. In addition, other anti-neoplastics such as gemcitabine, CDK inhibitor alvociclib and P450 inhibitor celecoxicib had highest predicted response signature scores in BN tumors.

LD subtype are androgen receptor (AR) driven tumors, have the highest expression levels of prostate terminal differentiation markers, lower metastatic potential and are sensitive to androgen deprivation therapy (ADT). LP subtype tumors are also AR driven, have the highest expression levels of proliferation markers, higher metastatic potential and are insensitive to ADT but sensitive to taxane-based chemotherapy and androgen receptor signaling inhibitors (ARSI). BI tumors, are non-AR driven tumors but have elevated expression of other sex steroid transcription factors such as the estrogen receptor, glucocorticoid receptor or progesterone receptors and are sensitive to ADT. BI tumors have the highest expression of markers of an activated tumor immune microenvironment, higher metastatic potential and are additionally sensitive to radiotherapy, protein kinase inhibitors and immune-checkpoint therapy. BN tumors are non-AR driven tumors, have the lowest expression of prostate terminal differentiation markers, highest expression of markers of a suppressed tumor immune microenvironment and are resistant to ADT but sensitive to platinum and vinca alkaloid chemotherapies. These results further showed that prostate subtyping classifiers of the disclosure predict response to cancer drugs.

These results showed that a genomic classifier of the disclosure could be used to identify four molecular subtypes in prostate cancer subjects. The results further showed that the molecular subtypes of the present disclosure have distinct clinical associations. These results suggested that the methods and markers of the disclosure are useful for diagnosing, prognosing, determining the progression of cancer, or predicting benefit from therapy in a subject having prostate cancer. These results further showed that the subtyping methods and genomic classifiers of the disclosure are useful for predicting benefit from androgen deprivation therapy (ADT), radiation therapy (RT), chemotherapy and/or treating a subject with prostate cancer. The results showed that the subtyping methods of the disclosure may be used to determine a course of treatment for a subject with prostate cancer.

What is claimed is:

1. A method comprising:
(a) optionally providing, obtaining, or having obtained a biological sample from a prostate cancer subject;
(b) performing or having performed an assay to detect or determine the presence and/or expression level of at least one or more target genes selected from Table 2 in a sample from a prostate cancer subject; and
(c) administering a treatment to the subject, wherein the treatment is selected from the group consisting of surgery, chemotherapy, radiation therapy, immunotherapy/biological therapy, hormonal therapy, and photodynamic therapy based at least in part on the presence and/or expression level of the at least one or more genes selected from Table 2.

2. A method comprising:
(a) optionally providing, obtaining, or having obtained a biological sample from a subject with prostate cancer;
(b) performing or having performed an assay to detect or determine the presence and/or expression level in the biological sample for a plurality of targets, wherein the plurality of targets comprises one or more genes selected from Table 2;
(c) subtyping or obtaining the subtype of the prostate cancer in the subject based on the presence or expression levels of the plurality of targets; and
(d) administering a treatment to the subject, wherein the treatment is selected from the group consisting of surgery, chemotherapy, radiation therapy, immunotherapy/biological therapy, hormonal therapy, and photodynamic therapy based at least in part on the subtype of the prostate cancer.

3. The method of claim 2, wherein the prostate cancer subtype is selected from the group comprising or consisting of Luminal Differentiated (LD), Luminal Proliferating (LP), Basal Immune (BI), and Basal Neuroendocrine-like (BN).

4. A method comprising:
(a) optionally providing, obtaining, or having obtained a biological sample from a subject having prostate cancer;
(b) measuring or obtaining a measure of the levels of expression in the biological sample of a plurality of target genes selected from Table 2; and
(c) subtyping the prostate cancer of the subject according to a genomic subtyping classifier based on the levels of expression of the plurality of target genes, wherein said subtyping comprises assigning the prostate cancer to a subtype selected from the group comprising or consisting of Luminal Differentiated (LD), Luminal Proliferating (LP), Basal Immune (BI), and Basal Neuroendocrine-like (BN).

5. The method of claim 4, further comprising prescribing a treatment regimen to the subject based at least in part on the prostate cancer subtype.

6. The method of claim 4 or 5, further comprising administering a treatment to the subject, based at least in part on the subtype of the cancer.

7. The method of any one of claims 4-6, wherein the treatment is selected from the group consisting of surgery, chemotherapy, radiation therapy, immunotherapy/biological therapy, hormonal therapy, and photodynamic therapy.

8. The method of any one of claims 1-7, wherein the expression level of said target is reduced expression of said target.

9. The method of any one of claims 1-7, wherein the expression level of said target is increased expression of said target.

10. The method of any one of claims 1-9, wherein the level of expression of said target is detected or determined by using a method selected from the group consisting of in situ hybridization, a PCR-based method, an array-based method, an immunohistochemical method, an RNA assay method and an immunoassay method.

11. The method of any one of claims 1-10, wherein said method comprises using a reagent selected from the group consisting of a nucleic acid probe, one or more nucleic acid primers, and an antibody.

12. The method of claim 11, wherein said nucleic acid probe is a reporter probe and/or a capture probe.

13. A method of treating a subject with prostate cancer, comprising:
(a) optionally providing, obtaining, or having obtained a biological sample comprising prostate cancer cells from the subject;
(b) performing or having performed an assay to determine or detect the presence and/or level of expression of at least one or more targets selected from Table 2 using at least one reagent that specifically binds to said targets;
(c) subtyping or obtaining the subtype of the prostate cancer based on the presence and/or level of expression of the at least one or more targets; and
(d) prescribing and/or administering a treatment regimen to the subject based at least in part on the prostate cancer subtype.

14. The method of claim 13, wherein the prostate cancer subtype is selected from the group consisting of Luminal Differentiated (LD), Luminal Proliferating (LP), Basal Immune (BI), and Basal Neuroendocrine-like (BN).

15. The method of any one of claims 13-14, wherein said reagent is selected from the group consisting of a nucleic acid probe, one or more nucleic acid primers, and an antibody.

16. The method of claim 15, wherein said nucleic acid probe is a reporter probe and/or a capture probe.

17. The method of any one of claims 13-16, wherein the treatment regimen is surgery, chemotherapy, radiation therapy, immunotherapy/biological therapy, hormonal therapy, and/or photodynamic therapy.

18. The method of any one of claims 1-17, wherein the chemotherapy is bortezomib, carfilzomib, alvespimycin, tanespimyicin, docetaxel, paclitaxel, dasatinib, erlotinib, gefitinib, ibrutinib, olaparib, pazopanib, vandetinib, staurosporine, hydroxy-staurosporine, rapamycin, everolimus, lovostatin, somastatin, carboplatin, cisplatin, oxaliplatin, campothecin, cyclosphosphamide, etoposide, ifosfamide, mitoxantrone, epirubicin, doxorubicin, vinorelbine, vincristine and vinblastine, gemcitabine, alvociclib, or celecoxicib.

19. The method of any one of claims 1-18, comprising subtyping the subject as having LD prostate cancer.

20. The method of claim 19, further comprising predicting the subject as benefiting from radiotherapy and/or radical prostatectomy, optionally benefiting more than a subject having LP, BI or BN prostate cancer.

21. The method of claim 19 or 20, further comprising prescribing and/or administering primary radiotherapy and/or radical prostatectomy.

22. The method of claim 19, further comprising predicting the subject as not benefiting from docetaxel in addition to ADT.

23. The method of claim 19 or 22, further comprising not prescribing and/or not administering docetaxel in addition to ADT.

24. The method of claim 19, further comprising characterizing the LD prostate cancer as one or more of: androgen receptor (AR) driven; having high expression levels of prostate terminal differentiation markers, optionally higher than LP, BI or BN prostate cancer; having lower metastatic potential, optionally wherein the metastatic potential is lower than LP and BI prostate cancer; and/or being sensitive to ADT.

25. The method of claim 19 or 24, further comprising prescribing and/or administering ADT.

26. The method of any one of claims 1-18, comprising subtyping the subject as having LP prostate cancer.

27. The method of claim 26, further comprising predicting the subject as benefiting from: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel.

28. The method of claim 26 or 29, further comprising prescribing and/or administering: a drug that regulates the proteasome or cellular protein metabolism, optionally bortezomib, carfilzomib, alvespimycin, or tanespimyicin; and/or a drug that inhibits cellular division through abrogation of the microtubule complexes, optionally docetaxel or paclitaxel.

29. The method of claim 26, further comprising predicting the subject as benefiting from docetaxel in addition to ADT.

30. The method of claim 26 or 29, further comprising prescribing and/or administering docetaxel in addition to ADT.

31. The method of claim 26, further comprising characterizing the LP prostate cancer as one or more of: androgen receptor (AR) driven; having high expression levels of proliferation markers, optionally higher than LD, BI and BN prostate cancer; having higher metastatic potential, optionally higher than LD prostate cancer, insensitive to ADT; and/or sensitive to taxane-based chemotherapy and androgen receptor signaling inhibitors (ARSI).

32. The method of claim 26 or 31, further comprising not prescribing and/or not administering ADT.

33. The method of claim 26 or 31, further comprising prescribing and/or administering a taxane-based chemotherapy and/or an ARSI.

34. The method of claim 26, further comprising predicting the subject as benefiting less from primary radiotherapy and/or radical prostatectomy than a subject with LD prostate cancer.

35. The method of claim 26, further comprising predicting the subject as benefiting more from primary radiotherapy than a subject with BN prostate cancer.

36. The method of any one of the preceding claims, comprising subtyping the subject as having LD prostate cancer, or, comprising subtyping the subject as having LP prostate cancer.

37. The method of claim 36, further comprising predicting the subject as not benefiting from higher dose primary radiotherapy, optionally wherein the higher dose primary radiotherapy is 79 Gy.

38. The method of claim 36 or 37, further comprising not prescribing and/or not administering a higher dose primary radiotherapy, optionally wherein the higher dose primary radiotherapy is 79 Gy.

39. The method of claim 36, 37 or 38, further comprising prescribing and/or administering a lower dose primary radiotherapy, optionally wherein the lower dose primary radiotherapy is 70 Gy.

40. The method of claim 36, further comprising predicting the subject as not benefitting from long term adjuvant ADT, optionally wherein the long term ADT is 28 months.

41. The method of claim 36 or 40, further comprising not prescribing and/or not administering long term adjuvant ADT, optionally wherein the long term ADT is 28 months.

42. The method of claim 36, 40 or 41, further comprising prescribing and/or administering short term adjuvant ADT, optionally wherein the short term ADT is 4 months.

43. The method of claim 36, further comprising predicting the subject as benefiting from the addition of long term ADT to salvage RT in combination with following biochemical recurrence, optionally wherein long term ADT is 24 months.

44. The method of claim 36 or 43, further comprising prescribing and/or administering long term ADT in addition to salvage RT following biochemical recurrence, optionally wherein long term ADT is 24 months.

45. The method of claim 36, further comprising predicting the subject as benefiting from abiraterone acetate.

46. The method of claim 36 or 45, further comprising prescribing and/or administering abiraterone acetate.

47. The method of any one of claims 1-18, comprising subtyping the subject as having BN prostate cancer.

48. The method of claim 47, further comprising predicting the subject as benefiting from: an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib.

49. The method of claim 47 or 48, further comprising prescribing and/or administering: an alkylating agent, optionally carboplatin, cisplatin, oxaliplatin, or campothecin; and/or a topoisomerase inhibitor, optionally cyclosphosphamide, etoposide, ifosfamide, mitoxantrone, epirubicin, or doxorubicin; and/or a vinca alkaloid, optionally vinorelbine, vincristine or vinblastine; and/or an anti-neoplastic, optionally gemcitabine, CDK inhibitor alvociclib or P450 inhibitor celecoxicib.

50. The method of claim 47, further comprising predicting the subject as not benefiting from docetaxel in addition to ADT.

51. The method of claim 47 or 50, further comprising not prescribing and/or not administering docetaxel in addition to ADT.

52. The method of claim 47, further comprising predicting the subject as not benefiting from abiraterone acetate.

53. The method of claim 47 or 52, further comprising not prescribing and/or not administering abiraterone acetate.

54. The method of claim 47, further comprising predicting the subject as benefiting less from primary radiotherapy and/or radical prostatectomy than a subject with LD prostate cancer.

55. The method of claim 47, further comprising predicting the subject as benefiting less from primary radiotherapy than a subject with LD, BI or LP prostate cancer.

56. The method of claim 47, further comprising characterizing the BN prostate cancer as one or more of: non-AR driven; having the low expression of prostate terminal differentiation markers, optionally lower than LD, LP and BI prostate cancer; high expression of markers of a suppressed tumor immune microenvironment, optionally higher than LD, LP and BI prostate cancer; being resistant to ADT; and/or being sensitive to platinum and vinca alkaloid chemotherapies.

57. The method of claim 47 or 56, further comprising prescribing and/or administering a platinum and/or a vinca alkaloid chemotherapy.

58. The method of claim 47 or 56, further comprising not prescribing and/or not administering ADT.

59. The method of any one of claims 1-18, comprising subtyping the subject as having BI prostate cancer.

60. The method of claim 59, further comprising predicting the subject as benefiting from: a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin.

61. The method of claim 59 or 60, further comprising prescribing and/or administering: a protein kinase inhibitor, optionally dasatinib, erlotinib, gefitinib, ibrutinib, olaparib, pazopanib, vandetinib, staurosporine or hydroxy-staurosporine; and/or an mTOR pathway inhibitor, optionally rapamycin or everolimus; and/or an HMG CoA inhibitor, optionally lovostatin or somastatin.

62. The method of claim 59, further comprising predicting the subject as not benefiting from docetaxel in addition to ADT.

63. The method of claim 59 or 62, further comprising not prescribing and/or not administering docetaxel in addition to ADT.

64. The method of claim 59, further comprising predicting the subject as benefiting less from primary radiotherapy and/or radical prostatectomy than a subject with LD prostate cancer.

65. The method of claim 59, further comprising predicting the subject as benefiting more from primary radiotherapy than a subject with BN prostate cancer.

66. The method of claim 59, further comprising characterizing the BI prostate cancer as one or more of: non-AR driven; having elevated expression of other sex steroid transcription factors, optionally estrogen receptor, glucocorticoid receptor and progesterone receptors; sensitive to ADT; having high expression of markers of an activated tumor immune microenvironment, optionally higher than LD, LP and BN prostate cancer; having higher metastatic potential, optionally higher than LD prostate cancer; and/or sensitive to radiotherapy, protein kinase inhibitors and immune-checkpoint therapy.

67. The method of claim 59 or 66, further comprising prescribing and/or administering ADT, radiotherapy, a protein kinase inhibitor and/or immune-checkpoint therapy.

68. The method of any one of 1-18, or 47-67, comprising subtyping the subject as having BN prostate cancer, or, comprising subtyping the subject as having BI prostate cancer.

69. The method of claim 68, further comprising predicting the subject as benefiting from higher dose primary radiotherapy, optionally wherein the higher dose primary radiotherapy is 79 Gy.

70. The method of claim 68 or 69, further comprising prescribing and/or administering a higher dose primary radiotherapy, optionally wherein the higher dose primary radiotherapy is 79 Gy.

71. The method of claim 68, 69 or 70, further comprising not prescribing and/or not administering a lower dose primary radiotherapy, optionally wherein the lower dose primary radiotherapy is 70 Gy.

72. The method of claim 68, further comprising predicting the subject as benefiting from long term adjuvant ADT, optionally wherein the long term ADT is 28 months.

73. The method of claim 68 or 72, further comprising prescribing and/or administering long term adjuvant ADT, optionally wherein the long term ADT is 28 months.

74. The method of claim 68, 72 or 73, further comprising not prescribing and/or not administering short term adjuvant ADT, optionally wherein the short term ADT is 4 months.

75. The method of claim 68, further comprising predicting the subject as not benefiting from the addition of long term ADT to salvage RT in combination with following biochemical recurrence, optionally wherein long term ADT is 24 months.

76. The method of claim 68 or 75, further comprising not prescribing and/or administering long term ADT in addition to salvage RT following biochemical recurrence, optionally wherein long term ADT is 24 months.

77. The method of any one of the preceding claims, wherein the sample or biological sample is a biopsy, urine sample, a blood sample or a prostate tumor sample.

78. The method of claim 77, wherein the blood sample is plasma, serum, or whole blood.

79. The method of any one of the preceding claims, wherein the subject is a human.

80. The method of any one of the preceding claims, wherein the level of expression is increased or reduced compared to a control.

81. The method of any one of the preceding claims, wherein said measuring the level of expression comprises measuring the level of an RNA transcript.

82. The method of any one of the preceding claims, wherein the plurality of targets are nucleic acid targets.

83. The method of claim 82, wherein the plurality of nucleic acid targets comprises a coding target.

84. The method of claim 83, wherein the coding target is an exonic sequence.

85. The method of any one of claims 82-84, wherein the plurality of nucleic acid targets comprises a non-coding target.

86. The method of claim 85 wherein the non-coding target comprises an intronic sequence or partially overlaps an intronic sequence.

87. The method of claim 85, wherein the non-coding target comprises an intergenic sequence.

88. The method of claim 85, wherein the non-coding target comprises a sequence within the untranslated region (UTR) or partially overlaps with a UTR sequence.

89. The method of any one of claims 82-88, wherein the plurality of nucleic acid targets comprise a DNA sequence.

90. The method of any one of claims 82-88, wherein the plurality of nucleic acid targets comprise an RNA sequence.

91. The method of any one of claims 82-90, further comprising sequencing the plurality of nucleic acid targets.

92. The method of any one of claims 82-90, further comprising hybridizing the plurality of nucleic acid targets to a solid support.

93. The method of claim 92, wherein the solid support is a bead or array.

94. A probe set for use in the method of any one of the preceding claims, the probe set comprising or consisting of a plurality of probes, wherein the probes in the set are used for detecting the expression level of a plurality of nucleic acid targets in a sample from the subject, wherein the plurality of nucleic acid targets comprise or consist of a plurality of targets selected the targets in Table 2, optionally wherein the probes of the probe set hybridize to the nucleic acid targets.

95. A probe set for subtyping, prognosing and/or predicting benefit from prostate cancer therapy of a prostate cancer in a subject comprising or consisting of a plurality of probes, wherein the probes in the set are used for detecting the expression level of a plurality of nucleic acid targets in a sample from the subject, wherein the plurality of nucleic acid targets comprise or consist of a plurality of targets selected the targets in Table 2, optionally wherein the probes of the probe set hybridize to the nucleic acid targets.

96. The probe set of claim 94 or 95, wherein the plurality of nucleic acid targets comprises a coding target.

97. The probe set of claim 96, wherein the coding target is an exonic sequence.

98. The probe set of any one of claims 94-97, wherein the plurality of nucleic acid targets comprises a non-coding target.

99. The probe set of claim 98, wherein the non-coding target comprises an intronic sequence or partially overlaps an intronic sequence.

100. The probe set of claim 98, wherein the non-coding target comprises an intergenic sequence.

101. The probe set of claim 98, wherein the non-coding target comprises a sequence within the untranslated region (UTR) or partially overlaps with a UTR sequence.

102. The probe set of any one of claims 94-101, wherein the plurality of nucleic acid targets comprise a DNA sequence.

103. The probe set of any one of claims 94-101, wherein the plurality of nucleic acid targets comprise an RNA sequence.

104. A system for analyzing a cancer, comprising:
(a) the probe set of any one of claims 94-103, and
(b) a computer model or algorithm for analyzing an expression level and/or expression profile of the target hybridized to the probe in a sample from a subject suffering from prostate cancer.

105. The system of claim 104, further comprising a computer model or algorithm for correlating the expression level or expression profile with disease state or outcome.

106. The system of claim 104, further comprising a computer model or algorithm for designating a treatment modality for the subject.

107. The system of claim 104, further comprising a computer model or algorithm for normalizing expression level or expression profile of the target sequences.

108. The method, probe set, or system of any one of the preceding claims, wherein the plurality of targets comprises at least about 2, at least about 3, at least about 4, at least about 5, at least about 6, at least about 7, at least about 8, at least about 9, or at least about 10 targets selected from Table 2.

109. The method, probe set, or system of any one of the preceding claims, wherein the plurality of targets comprises or consists of at least 2, 3, 4, 5, 6, 7, 8, 9, 10, 11, 12, 13, 14, 15, 20, 25, 30, 40, 50, 60, 70, 80, 90, 100, 110, 150, 200, 210 or 215 targets selected from the targets listed in Table 2, 2.1, 2.2, 2.3, 2.4, 2.5, 2.6, 2.7, 2.8, 2.9, 2.10, or 2.11.

110. The method, probe set, or system of any one of the preceding claims, wherein the plurality of targets does not include one or more of the targets listed in Table 2.

111. The method, probe set, or system of any one of the preceding claims, wherein the plurality of targets includes not more than 215, 210, 200, 175, 150, 125, 110, 100, 90, 80, 70, 60, 50, 40, 30, 20, 15, 14, 13, 12, 11, 10, 9, 8, 7, 6, or 5 of the targets listed in Table 2, 2.1, 2.2, 2.3, 2.4, 2.5, 2.6, 2.7, 2.8, 2.9, 2.10, or 2.11.

112. The method, probe set, or system of any one of the preceding claims, wherein the plurality of targets comprises or consists of targets having an absolute value of the coefficient value in Table 2 of at least 0.10000, 0.12500, 0.15000, 0.17500, 0.20000, 0.22500, 0.25000, 0.27500, 0.30000, 0.32500, 0.35000, 0.37500, 0.40000, 0.42500, 0.45000, 0.47500, 0.50000, 0.52500, 0.55000, 0.57500, 0.60000, 0.62500, 0.65000, 0.67500, 0.70000, 0.72500, 0.75000, 0.77500, 0.80000, 0.82500, 0.85000, 0.87500, 0.90000, 0.92500, 0.95000, 0.97500, 1.00000, 1.50000, 2.00000, 2.50000, 3.00000, 3.50000, or 4.00000.
